(19) United States
(12) Patent Application Publication
(10) Pub. No.: US 2024/0207506 A1
(43) Pub. Date: Jun. 27, 2024

(54) INSULIN INJECTION PUMP, MANUFACTURING METHOD OF INSULIN INJECTION PUMP AND CLOSED-LOOP SYSTEM

(71) Applicant: Peking University, Beijing (CN)

(72) Inventor: Yue CUI, Beijing (CN)

(21) Appl. No.: 18/396,507

(22) Filed: Dec. 26, 2023

(30) Foreign Application Priority Data
Dec. 27, 2022 (CN) 202211685429.X
Dec. 27, 2022 (CN) 202223498972.7
Dec. 27, 2022 (CN) 202223499937.7

(51) Int. Cl. A61M 5/14; A61M 5/145; A61M 5/172; B33Y 80/00

(52) U.S. Cl. CPC A61M 5/1413; A61M 5/14586; A61M 5/1723; A61M 2205/0294; A61M 2205/14; A61M 2205/3317; A61M 2207/00; A61M 2230/201; B33Y 80/00

(57) ABSTRACT

An insulin injection pump, a manufacturing method of an insulin injection pump and a closed-loop system is provided. The insulin injection pump includes an upper housing and a lower housing. A film is arranged between the upper housing and the low housing. An insulin storage chamber is formed between the film and the upper housing. Several conical holes are formed in the film. Large-diameter ends of the conical holes are adjacent to the insulin storage chamber. A piezoelectric ring is arranged on a side of the film facing away from the insulin storage chamber. The edges of two opposite sides of the film and the piezoelectric ring are connected with an external alternating current power supply through wires, respectively. A liquid outlet is formed in the lower housing.

### CROSS-REFERENCE TO RELATED APPLICATION

This patent application claims the benefit of and priority to Chinese Patent Application No. 202223499937.7, filed with the Chinese Patent Office on Dec. 27, 2022, Chinese Patent Application No. 202211685429.X, filed with the Chinese Patent Office on Dec. 27, 2022, and Chinese Patent Application No. 202223498972.7, filed with the Chinese Patent Office on Dec. 27, 2022, each of which is hereby incorporated by reference herein in its entirety.

### TECHNICAL FIELD

The present disclosure relates to the technical field of medical devices, in particular to an insulin injection pump, a manufacturing method of an insulin injection pump, and a closed-loop system.

### BACKGROUND

Diabetes mellitus refers to a group of metabolic diseases characterized by hyperglycemia. Hyperglycemia is caused by defects in insulin secretion or impaired biological actions, or both. Diabetes mellitus suffers from a long-standing hyperglycemia symptom which can lead to chronic damage and dysfunction of various tissues, especially eyes, kidneys, heart, blood vessels, and nerves.

In order to provide better and more portable control of blood sugar and improve the quality of life of diabetic patients, a diabetes closed-loop system came into being in the market to simulate the function of human pancreas to realize the automatic delivery of insulin. However, the current commercial diabetes closed-loop system is large in size and high in price, and is difficult for diabetic patients to carry and use widely. Thus, it is desired to develop a small-size, convenient-to-carry and low-price diabetes closed-loop system so that diabetic patients can maintain blood sugar balance for a long time to improve the quality of life.

### SUMMARY

The purpose of the present disclosure is to provide an insulin injection pump, a manufacturing method of an insulin injection pump, and a closed-loop system so as to solve the problems in the prior art. The insulin injection pump is small in size, simple in structure, convenient to carry and low in cost.

In order to achieve the purpose, the present disclosure provides the following solution.

The present disclosure provides an insulin injection pump, including an upper housing and a lower housing. A film is arranged between the upper housing and the low housing. An insulin storage chamber is formed between the film and the upper housing. Several conical holes are formed in the film. Large-diameter ends of the conical holes are adjacent to the insulin storage chamber. A piezoelectric ring is arranged on a side of the film facing away from the insulin storage chamber. The edges of two opposite sides of the film and the piezoelectric ring are connected with an external alternating current power supply through wires, respectively. A liquid outlet is formed in the lower housing. The film and the piezoelectric ring are arranged in such a way that the piezoelectric ring vibrates radially after being energized and drives the film to vibrate radially along with it, so that the conical holes in the film continuously stretch and bent. Therefore, insulin flows downwards in the conical holes, and insulin is pushed out and enters human body through an injection tube connected with the liquid outlet in the lower housing. Thus, the overall structure of the insulin injection pump is simple, the production cost is low, miniaturization production can be realized, and the insulin injection pump is convenient to carry.

Optionally, the film is made of a hard material or a soft material, and specifically, which can be selected from one of the following: stainless steel, gold, copper, zinc, platinum, silver, tungsten, aluminum, aluminum alloy, natural rubber, isoprene rubber, polybutadiene rubber, styrene-butadiene rubber, nitrile rubber, chloroprene rubber, butyl rubber, halogenated butyl rubber, ethylene-propylene methylene rubber, ethylene propylene diene methylene rubber, epichlorohydrin rubber, polyacrylate rubber, silicone rubber, fluorosilicone rubber, fluororubber, chlorosulfonated polyethylene, hydrogenated nitrile rubber, thermoplastic polyolefin elastomer, thermoplastic styrene elastomer, polyurethane thermoplastic elastomer, polyester thermoplastic elastomer, polyamide thermoplastic elastomer, halogen-containing thermoplastic elastomer, ionic thermoplastic elastomer, ethylene copolymer thermoplastic elastomer, 1,2-polybutadiene thermoplastic elastomer, trans-polyisoprene thermoplastic elastomer, melt-processed thermoplastic elastomer, thermoplastic vulcanizate, and polydimethylsiloxane. The film can be made of metal, rubber or elastomer material, so that the film is of a certain degree of flexibility and will continuously stretch and bent.

Optionally, the piezoelectric ring is made of a piezoelectric crystal, a piezoelectric ceramic or a piezoelectric polymer. Piezoelectric crystals, piezoelectric ceramics and piezoelectric polymer are piezoelectric materials. The piezoelectric ring vibrates radially after being connected to an alternating current, and ultrasonic waves are induced by the vibration. The piezoelectric ring is made of piezoelectric material, so that on one hand, the vibration of the piezoelectric ring drives the film to vibrate to administer insulin, and on the other hand, the induced ultrasonic waves can promote the absorption of insulin by human body.

Optionally, insulin, with the concentration of 1-500 U/ml, is stored in the insulin storage chamber.

The voltage of the external alternating current power supply connected with the film and the piezoelectric ring is 10 V to 100 V. The piezoelectric ring vibrates, and ultrasonic waves are induced by the vibration to promote the absorption of insulin by human body. The piezoelectric ring and the film are connected into a whole, so that the vibration of the piezoelectric ring directly drives the vibration of the film to avoid energy loss.

Optional, sealing rings are arranged between the film and the upper housing and between the piezoelectric ring and the lower housing. The sealing rings are arranged between the film and the upper housing and between the piezoelectric ring and the lower housing, so that the connection between these components is closer, the sealing performance is high, and avoiding the leakage of medicine. A liquid inlet is formed in the upper housing, and a rubber plug is arranged at the liquid inlet. The liquid inlet is formed in the housing and is tightly plugged by the rubber plug, so that the insulin stored in the insulin storage chamber can be supplied after used up, and realizing the cyclic utilization of the pump body.

The present disclosure also provides a manufacturing method of an insulin injection pump, including the following steps:
S1, preparing a film: taking a sheet with a thickness of 1-5 mm, and forming conical holes by laser etching or ion selective etching therein;
S2, preparing a piezoelectric ring: selecting piezoelectric materials, and performing sputtering deposition of a layer of piezoelectric materials through mask holes at an outer edge of the film to form a ring which is integrated with the film and has a same outer diameter as an outer diameter of the film;
S3, preparing an upper housing: integrally molding an upper housing through 3D (three-dimensional) printing or injection molding;
S4, preparing a lower housing: integrally molding a lower housing through 3D printing or injection molding; and
S5, assembling: sequentially placing a sealing ring, the film, the piezoelectric ring and a sealing ring in the upper housing to seal and connect the lower housing with the upper housing. The conical holes can be formed by laser etching or ion selective etching, so that it is convenient to form the holes with high aperture accuracy and smooth surfaces inside the holes, which facilitates the flow of insulin in the holes. The film, the piezoelectric ring, the upper housing, and the lower housing are assembled after being prepared, and the sealing rings are arranged between the film and the upper housing and between the piezoelectric ring and the lower housing, so that the connection between these components is sealed to ensure the best administration effect. Sputtering deposition of a layer of piezoelectric materials is performed on the film through mask holes to form the piezoelectric ring, so that the film and the piezoelectric ring are formed as a whole. The piezoelectric ring vibrates after being energized, and the vibration can be effectively transmitted to the film to drive the film to vibrate together, so that transmission loss is reduced.

The present disclosure provides a closed-loop system, including a sensor, a control unit and the insulin injection pump as described above. The sensor includes electrodes. The sensor is arranged at a position of a liquid outlet of the insulin injection pump. An input end of the control unit is connected with an output end of the sensor, and an output end of the control unit is connected with an input end of the insulin injection pump. The control unit controls the start and stop of the insulin injection pump after receiving an electrical signal from the sensor. The insulin injection pump is integrated with the sensor, and a closed loop is formed though the connection and control of the control unit. The glucose concentration in interstitial fluid of the patient is detected by the electrodes of the sensor so as to control the operation of the insulin injection pump. The sensor structure is used as an insulin injection channel to realize the automatic detection of blood glucose and the automatic delivery of insulin for diabetic patients, so that the functions of detection and treatment are integrated. The insulin injection pump is small in size, convenient to carry and low in cost, and can meet the requirements of automatic detection of blood glucose and automatic delivery of insulin in daily life of diabetic patients, so that the quality of life is improved.

Optionally, the sensor is a hose sensor, the hose sensor includes a tubular base, and the electrodes are circumferentially distributed on an outer wall of the base. The cross section of the tubular base is circular or polygonal, and the length of the tubular base is 1 mm to 15 mm. When the cross section of the tubular base is circular, the electrodes are arranged on the outer wall of the tubular base, and when the cross section of the tubular base is polygonal, an electrode can be arranged on each polygonal surface of the tubular base.

Optionally, the sensor is a microneedle biosensor, and includes a substrate. A microneedle array is integrally formed on the substrate. The microneedle array includes several hollow microneedles. The substrate is covered with the electrodes. Each hollow microneedle is conical or pyramid-shaped, and a large-diameter end of each hollow microneedle is connected with the substrate. Each hollow microneedle is conical-shaped, and a tip of each hollow microneedle is easy to penetrate into the skin.

The electrodes include a working electrode and an active electrode. The working electrode and the active electrode are arranged to form a loop, and the active electrode can play the role of circuit connection and voltage stabilization. Alternatively, the electrodes include a working electrode, a counter electrode and a reference electrode. The working electrode, the counter electrode and the reference electrode are arranged in such a way that the counter electrode plays the role of circuit connection, and the reference electrode plays the role of voltage stabilization.

Optionally, the control unit includes a signal acquisition module, a control module and an execution module. The signal acquisition module is used for receiving and converting electrical signals of the sensor. The control module analyzes the electrical signal collected by the signal acquisition module and issues instructions to the execution module. The execution module controls the start and stop of the insulin injection pump according to the instructions sent by the control module. The signal acquisition module, the control module and the execution module are arranged to form a closed loop of the control system through acquisition, analysis and execution.

Compared with the prior art, the present disclosure has the following technical effects.

Firstly, in the present disclosure, the upper housing, the lower housing, the insulin storage chamber, the film, and the piezoelectric ring are arranged in such a way that the piezoelectric ring vibrates radially after being energized and drives the film to vibrate radially along with it, so that the conical holes in the film continuously stretch and bent. Therefore, insulin in the insulin storage chamber flows downwards in the conical holes, and insulin is pushed out and enters human body through an injection tube connected with the liquid outlet in the lower housing. Thus, the overall structure of the insulin injection pump is simple, the production cost is low, miniaturization production can be realized, and the insulin injection pump is convenient to carry and wearable.

Secondly, in the present disclosure, the conical holes can be formed by laser etching or ion selective etching, which facilitates the formation of holes with high aperture accuracy and smooth surfaces inside the holes, and facilitates the flow of the insulin inside the holes. The film, the piezoelectric ring, the upper housing and the lower housing are assembled after being manufactured, and the sealing rings are arranged between the film and the upper housing and between the piezoelectric ring and the lower housing, so that the connections between these components are sealed to ensure the optimal administration effect.

Thirdly, in the present disclosure, a layer of piezoelectric material is sputter deposited on the film through mask holes to form the piezoelectric ring, so that the film and the piezoelectric ring are formed as a whole. The piezoelectric ring vibrates after being energized, and the vibration can be effectively transmitted to the film to drive the film to vibrate together, thereby reducing transmission loss.

Fourthly, in the present disclosure, the insulin injection pump is integrated with the sensor, and a closed loop is formed though the connection and control of the control unit. The glucose concentration in interstitial fluid of the patient is collected and detected by the sensor so as to control the operation of the insulin injection pump. The automatic detection of blood glucose and the automatic delivery of insulin for diabetic patients are realized, so that the functions of detection and treatment are integrated. The insulin injection pump is small in size, wearable, convenient to carry and low in cost, and can meet the requirements of automatic detection of blood glucose and automatic delivery of insulin in daily life of diabetic patients, so that the quality of life is improved.

### BRIEF DESCRIPTION OF THE DRAWINGS

To more clearly illustrate the present embodiment of the present disclosure or the technical solution in the prior art, the following briefly introduces the attached figures to be used in the embodiments. Apparently, the attached figures in the following description show merely some embodiments of the present disclosure, and those skilled in the art may still obtain other drawings from these attached figures without creative efforts.

FIG. 1 is an integral structural schematic diagram of an insulin injection pump in the first embodiment of the present disclosure.

FIG. 2 is an integral structural exploded view of an insulin injection pump in the first embodiment of the present disclosure.

FIG. 3 is a cross-sectional schematic diagram of an insulin injection pump in the first embodiment of the present disclosure.

FIG. 4 is a schematic diagram of stretching and squeezing of a film and conical holes in the first embodiment of the present disclosure.

FIG. 5 is a schematic diagram of stretching of a film in the first embodiment of the present disclosure.

FIG. 6 is a schematic diagram of bending of a film in the first embodiment of the present disclosure.

FIG. 7 is a flow diagram of a manufacturing method of an insulin injection pump in the first embodiment of the present disclosure.

FIG. 8 is a flow diagram of a closed-loop system in the second embodiment of the present disclosure.

FIG. 9 is a structural schematic diagram of a closed-loop system in the second embodiment of the present disclosure.

FIG. 10 is a structural schematic diagram of a tubular base of a hose sensor in a bending state in the second embodiment of the present disclosure.

FIG. 11 is a structural schematic diagram of an insulin injection pump in the second embodiment of the present disclosure.

FIG. 12 is a structural schematic diagram of a circular tubular hose sensor with two electrodes in the second embodiment of the present disclosure.

FIG. 13 is a structural schematic diagram of a circular tubular hose sensor with three electrodes in the second embodiment of the present disclosure.

FIG. 14 is a cross-sectional view of a rectangular tubular hose sensor with two electrodes in the second embodiment of the present disclosure.

FIG. 15 is a cross-sectional view of a hexagonal tubular hose sensor with three electrodes in the second embodiment of the present disclosure.

FIG. 16 is a flow diagram of a closed-loop system in the third embodiment of the present disclosure.

FIG. 17 is a structural schematic diagram in the third embodiment of the present disclosure.

FIG. 18 is a structural schematic diagram of a microneedle biosensor with two electrodes in the third embodiment of the present disclosure.

FIG. 19 is a structural schematic diagram of a microneedle biosensor with three electrodes in the third embodiment of the present disclosure.

FIG. 20 is a structural schematic diagram of a microneedle biosensor with a pyramidal hollow microneedle in the third embodiment of the present disclosure.

FIG. 21 shows the principle and structural schematic of a microneedle based closed-loop system for the treatment of diabetes, wherein (a) is a schematic illustration of the closed-loop principle for controlling blood glucose (BG); (b) is a schematic illustration of the structure of the closed-loop system in interstitial fluid with the microneedles for detecting glucose and delivering insulin; (c) shows the components of the microneedles consist of the detecting microneedles and the delivering microneedles; (d) is a photograph of the way of managing diabetes with the closed-loop system by transferring the BG data to a phone App with a Bluetooth and a doctor by a cloud storage; (e) is a structural diagram of the closed-loop system, including the microneedles (red), PZT (white), steel sheet (purple), and insulin storage reservoir (blue); (f) is a photograph of the microneedle sensor; (g) is a photograph of the closed-loop system worn on a person's arm; (h) is a flow diagram of the closed-loop system with the function of powering and controlling the CGM and the pump.

FIG. 22 shows the fabrication and characterization of the sensing electrode, wherein (a) is a schematic diagram of the process of manufacturing the microneedle sensor; (b) is a SEM image of the front and back (illustration) of the microneedle electrode; (c) shows the loss-weight rate of the PLA microneedles immersed in the buffer at pH 7.5 at room temperature; (d) is a principle schematic of inducing micro-holes on the electrode by electrolyzing H₂O; (e) is a SEM image of the Au electrode, O—Au (top) and N—Au after the electrolysis (bottom); (f) is a AFM image of the Au surface after the electrolysis (top) and before the electrolysis (bottom); (g) shows the roughness of the Au surface after various electrolysis times; (h) shows the CV curves of Au/PB electrode with various electrolysis time, solution: 50 mM PBS and 2.5 mM Fe₆^{3-/4-}; (i) shows the EIS of Au/PB electrode with various electrolysis times, solution: 50 mM PBS and 2.5 mM Fe₆^{3-/4-}; (j) is a infrared image of the microneedle electrodes, including N—Au/PB (red line), O—Au/PB (blue line) and O—Au (black line); (k) shows the current response of N—Au/PB and O—Au/PB electrode to 4 mM H₂O₂ in the buffer.

FIG. 23 shows the electrochemical characterization of the microneedle sensor, wherein (a) shows the structure of the sensor and the electrochemical reaction; (b) shows the baseline current and the calibration curve (inset) of the microneedle sensor for detecting H₂O₂ in buffer; (c) shows the current-time curve for detecting glucose with various concentrations in buffer; (d) shows the current responses to some interferences, including lactate (green), uric acid (purple), dopamine (blue), ascorbate (yellow), and glucose (red); (e) shows the relative current response to 2 mM glucose at a buffer pH ranging from 6 to 8; (f) shows the relative current baseline of 2 mM glucose at various temperatures; (g) shows the relative current baselines to 2 mM glucose with an increasing number of tests; (h) shows the relative current baselines in response to glucose varied with different bending times at 30° C.; (i) shows the relative current baselines in response to glucose after bending at different angles.

FIG. 24 shows the structure, principle, and electrical characteristics of the ultrasonic pump, wherein (a) is a schematic diagram of a 3D model of an ultrasonic pump; (b) is a schematic diagram of the conical hole driving insulin, stretching and extruding to push insulin with AC; (c) is a photo of the ultrasonic insulin pump and the driving circuit board (inset); (d) is a SEM image of conical micro-pore on steel sheet; (e) is an enlarged view of the front and back of the conical micro-pore (illustration); (f) shows the impedance of the ultrasonic pump at different frequencies detected by the impedance analyzer; (g) shows the vibration frequency measured by the oscilloscope and the current-power curve (illustration) of the ultrasonic pump; (h) shows the flow rates of the ultrasonic pump at various powers; (i) shows the flow rates of the ultrasonic pump with various insulin concentrations; (j) is a schematic diagram of sound flow, microbubble cavitation, and induced perforation enhancing insulin diffusion; (k) is a illustration of the experiment process for the insulin diffusion at the simulated environment; (l) is a fluorescence image of the diffusing insulin in simulated interstitial fluids, with an ultrasonic pump (right) and without an ultrasonic pump (left).

FIG. 25 shows the in-vivo treatment of diabetic rats by the microneedle closed-loop system, wherein (a) is a photo of the rat's experiment with the closed-loop system fixed on a rat, including the circuit board and software; (b) shows a working method of the closed-cool system of alternating the blood glucose (BG) detection (yellow line) and the insulin delivery (purple line) with each cycle for 10 min; (c) shows the changing of blood glucose detected by a glucometer and the current measured by the microneedle sensor over 4 h in a rat; (d) is a Clark error grid, the BG differences from approximately 360 data between the predicted values detected by the microneedle sensor and the reference value detected by a glucometer; (e) shows the changing BG-time curve in diabetic rats using ultrasound to inject insulin to reduce BG, in comparison with the control group of the saline injection, no any treatment and the microneedle insertion; (f) shows the BG-time of the continuous subcutaneous insulin injection (CSII) with ultrasound (top), the one single injection (OSI) without ultrasound (middle) and the one single injection with ultrasound (bottom); (g) shows the experiment process of a simulating meal for managing rats' diabetes by the closed-loop system; (h) shows the changing blood glucose-versus-time curve with the closed-loop function (blue line) and without the closed-loop function (red line); (i) illustrates the test for the meal, the changing BG-time curve with the closed-loop function (blue line) and without the closed-loop function (red line), indicating the ability to control blood glucose by the closed-loop system.

FIG. 26 shows the current-time curve of glucose detection with two electrodes and three electrodes, wherein N—Au/PB was worked as working electrode and AgCl/AgCl as reference electrode/counter electrode in the two-electrodes system; in the three-electrodes system, an additional Ti electrode was added as the counter electrode.

FIG. 27 shows the camera and optical images of the sensing electrodes on the PLA microneedles; wherein (a) is a camera image of the flexible PLA microneedles with bending at a large angle; (b) is a camera image of the PLA microneedles with a working electrode (Au/PB, bottom section of the sample) and a reference electrode (Ag/AgCl, top section of the sample); (c) is an optical image of the flat surface around the microneedles in Au/PB; (d) is an optical image of the flat surface around the microneedles in Ag/AgCl.

FIG. 28 is a schematic diagram of the closed-loop system, wherein the circuits include the detection circuit and the drug administration circuit.

FIG. 29 is a SEM of the cross-sectional area of a PLA microneedle, wherein the cross-sectional SEM of a hollow microneedle after clipping at 100 μm from the needle tip in height and the enlarged drawing of that.

FIG. 30 is a three-dimensional structure diagram showing the size of the microneedle, wherein the figure on the right is the structure diagram of the microneedle, and the picture on the lower left is the top view of the needle.

FIG. 31 shows the shape of the microneedle before and after insertion into the skin, wherein (a) is a shape of a microneedle before insertion; (b) is a shape of a microneedle after insertion. The image proves that the microneedle does not break after insertion into the skin.

FIG. 32 shows the electrochemical characterization of the reference electrode after chlorination; wherein (a) shows the relative potential of Ag/AgCl in this work using the open-circuit voltage for testing; (b) shows a comparison using the present work's Ag/AgCl and a commercial solid Ag/AgCl electrode for 2 mM H₂O₂ solution in 50 mM phosphate buffer solution at pH 7.5.

FIG. 33 is an optical image of the N—Au/PB surface.

FIG. 34 is a AFM of the Au electrode surface with different etching times, wherein (a) 0 s, (b) 5 s, (c) 10 s, (d) 15 s, (e) 20 s, and (f) 25 s.

FIG. 35 shows the impedance of the Au/PB electrode at different electrolysis times. Solution: 50 mM PBS and 2.5 mM Fe₆^{3-/4-}.

FIG. 36 shows the current versus time curves and the calibration curves of the MNS sensor for H₂O₂ at different voltages, wherein (a) 0.6 V, (b) 0.1 V, (c) 0 V, and (d) −0.1 V, and the calibration curves of that.

FIG. 37 shows the electrochemistry characterization of the CV, wherein (a) shows the CV curve of the N—Au/PB with 2 mM glucose in the buffer; (b) shows the calibration between the square roots of the scan rate and the peak current.

FIG. 38 shows the current of the sensor for repeatability of glucose detection, The sensors were detected for 20 mM glucose in the buffer, wherein (a) shows the current for the different samples of the sensor; (c) shows the current of sensors for repeatability test; (b) shows the current for the different samples of the sensor.

FIG. 39 shows the current response of the microneedle sensor after repeated bending, wherein Glucose: 2 mM, Angle: 60°. The current of the sensor did not change significantly after repeated bending. The reason for the noise in the sensing response was the change of the contact area between the buffer solution and the electrode after bending, or the change of the buffer's internal resistance.

FIG. 40 is an optical image of a stained rat skin after inserting and removing the microneedles, wherein (a) is an optical image of the skin before inserting microneedles; (b) is an optical image of the skin after inserting microneedles. The picture exhibited the broken epidermal layer in the shape of the needle (arrows pointed), indicating that the microneedles had penetrated the epidermis and entered interstitial fluid.

FIG. 41 shows the experimental setup of the closed-loop system on a rat, wherein the inset shows the indentation on the rat's skin after removing the microneedles.

FIG. 42 shows the current response of the microneedle sensor by applying a force of 0.8 N to the rat's skin vertically and removing the force. It showed a small current change with an extrusion on the rat skin, indicating that the sensor had a good flexibility and electrical connection with the skin.

FIG. 43 shows the error distribution of predicted blood glucose (BG) from 360 data in the range of 1-14 mM or 1%-40%. Although the largest error was about 40%, it mainly happened at the predicted high BG, and it had no bad effect for managing diabetes and would not introduce a wrong treatment project, as shown in FIG. 25(d). In the boxplot, the mean value of the predicted error was 1.4 mM; the median was 1.15 mM; the standard deviation was 1.61 mM; the maximum was 14 mM. The orange points were the entire error data of the predicted BG levels, showing most errors located within a small range. The inset on the upper-right illustrated the distribution of relative error over the entire range, and the mean relative error was 9.9%.

FIG. 44 shows the effect of ultrasound on lowering the blood glucose levels in diabetic rats after a one-time administration with an ultrasonic pump (blue line) and without an ultrasonic pump (red line).

FIG. 45 shows the effects of a closed-loop system and an injection of glucose on blood glucose, wherein the first step: the closed-loop system was using to reduce BG to normal value; the second step: a 3 ml of 10% saline glucose was injected into the rat to increase BG above a normal concentration; the third step: the closed-loop system worked again to reduce BG to a normal concentration.

FIG. 46 shows the blood glucose in healthy rats using closed-loop system after injecting glucose. The healthy rats were subcutaneously injected with glucose, and then the blood glucose was timely adjusted to reach the normal range with the automatic blood glucose management of the closed-loop system. It shows that the closed-loop system can effectively manage the blood glucose of healthy rats.

Reference signs: 1000, insulin injection pump; 10, upper housing; 11, liquid inlet; 12, rubber plug; 20, lower housing; 21, liquid outlet; 30, film; 31, conical hole; 40, insulin storage chamber; 50, piezoelectric ring; 60, sealing ring; 70, hose sensor; 71, tubular base; 80, microneedle biosensor; 81, substrate; 82, microneedle array; 821, hollow microneedle; 90, electrode; 91, working electrode; 92, active electrode; 93, counter electrode; 94, reference electrode; 100, control unit; 101, signal acquisition module; 102, control module; and 103, execution module.

### DETAILED DESCRIPTION OF THE EMBODIMENTS

The following clearly and completely describes the technical solution in the embodiments of the present disclosure with reference to the embodiments of the present disclosure. Apparently, the described embodiments are merely a part rather than all of the embodiments of the present disclosure. Based on the embodiments in the present disclosure, all other embodiments obtained by those skilled in the art under the premise of without creative efforts fall into the protection scope of the present disclosure.

The purpose of the present disclosure is to provide an insulin injection pump, a manufacturing method of an insulin injection pump, and a closed-loop system so as to solve the problems in the prior art. The insulin injection pump is small in size, simple in structure, convenient to carry and low in cost.

To make the foregoing purpose, features and advantages of the present disclosure clearer and more comprehensible, the present disclosure is further described in detail below with reference to the attached figures and specific embodiments.

### Embodiment I

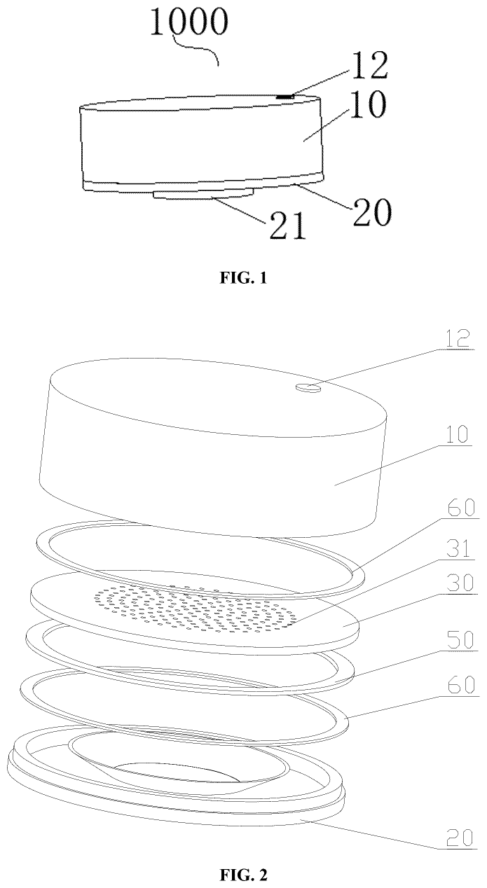

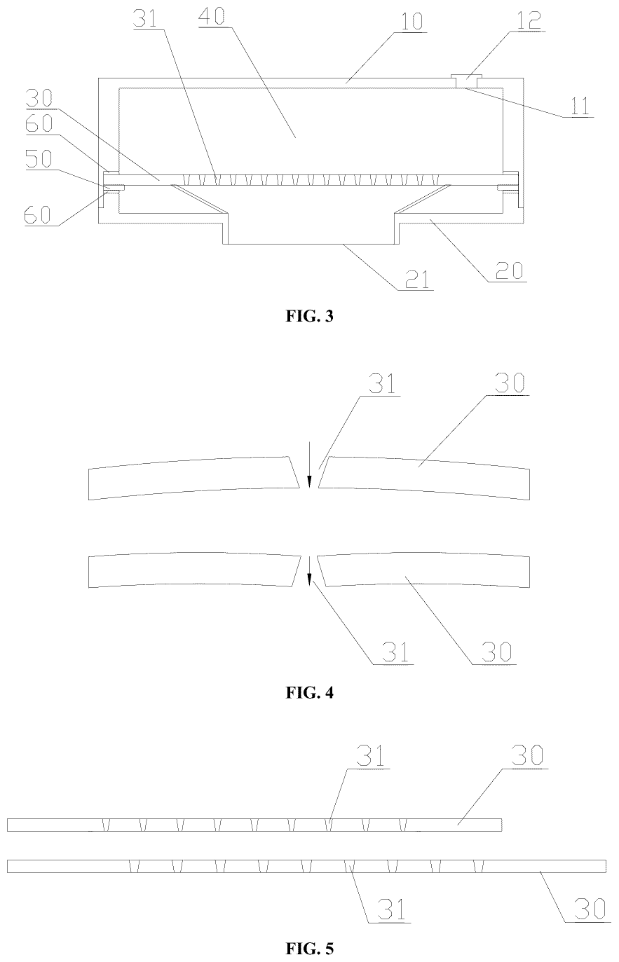

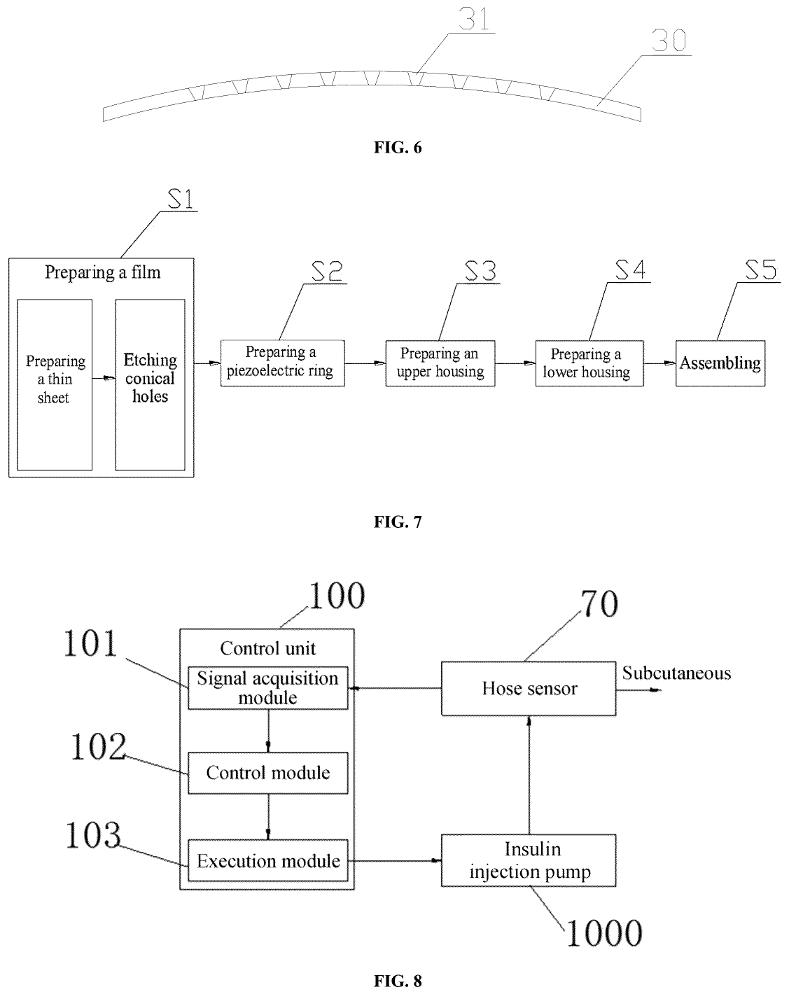

As shown in FIG. 1 to FIG. 7, the embodiment of the present disclosure discloses an insulin injection pump 1000, includes an upper housing 10 and a lower housing 20. A sealing ring 60, a film 30, a piezoelectric ring 50 and a sealing ring 60 are sequentially arranged between the upper housing 10 and the lower housing 20 from top to bottom. A liquid outlet 21 is formed in the bottom of the lower housing 20.

The film 30 is made of metal, rubber or elastomer, and specifically, which can be selected from one of the following: stainless steel, gold, copper, zinc, platinum, silver, tungsten, aluminum, aluminum alloy, natural rubber, isoprene rubber, polybutadiene rubber, styrene-butadiene rubber, nitrile rubber, chloroprene rubber, butyl rubber, halogenated butyl rubber, ethylene-propylene methylene rubber, ethylene propylene diene methylene rubber, epichlorohydrin rubber, polyacrylate rubber, silicone rubber, fluorosilicone rubber, fluororubber, chlorosulfonated polyethylene, hydrogenated nitrile rubber, thermoplastic polyolefin elastomer, thermoplastic styrene elastomer, polyurethane thermoplastic elastomer, polyester thermoplastic elastomer, polyamide thermoplastic elastomer, halogen-containing thermoplastic elastomer, ionic thermoplastic elastomer, ethylene copolymer thermoplastic elastomer, 1,2-polybutadiene thermoplastic elastomer, trans-polyisoprene thermoplastic elastomer, melt-processed thermoplastic elastomer, thermoplastic vulcanizate and polydimethylsiloxane. The film 30 can be made of hard film materials or flexible film materials, and in the embodiment flexible film materials are preferred, so that the film 30 is of a certain degree of flexibility and will continuously stretch and bent.

Several conical holes 31 are formed in the film 30. Large-diameter ends of the conical holes are formed in the side of the upper housing 10, and small-diameter ends of the conical holes are formed in the side of the lower housing 20. Specifically, the large-diameter ends of the conical holes 31 are larger than the molecular diameter of insulin, and the small-diameter ends of the conical holes 31 are smaller than the molecular diameter of insulin, so that insulin can enter the conical holes 31 but cannot automatically flow out. The conical holes 31 can be formed by laser etching or ion selective etching, so that it is convenient to form the holes with high aperture accuracy and smooth in surfaces inside the holes, which facilitates the flow of insulin in the holes.

The area between the film 30 and the upper housing 10 forms an insulin storage chamber 40, and the insulin storage chamber 40 is used for storing medicine to be injected. In the embodiment, the medicine stored in the insulin storage chamber 40 is insulin, with the concentration of 1-500 U/ml, which is used for the injection of insulin for diabetic patients.

A piezoelectric ring 50 is arranged on a side of the film 30 facing away from the insulin storage chamber 40. The piezoelectric ring 50 is made of piezoelectric crystals, piezoelectric ceramics or piezoelectric polymers. The edges of two opposite sides of the film 30 and the piezoelectric ring 50 are connected with an external alternating current power supply through wires, respectively. The voltage of the external alternating current power supply is 10 V to 100 V. Also, an external direct current of 1 to 10 V can be used, and is converted into alternating current of 10 V to 100 V through a circuit. The piezoelectric ring vibrates radially after being connected to alternating current, so that the film 30 is driven to vibrate. When the film 30 vibrates, the film 3 continuously stretch and bent, so that the conical holes 31 stretch, and the aperture thereof changes, that is, the large-diameter end and the small-diameter end of the conical holes 31 change continuously, the large-diameter end becomes smaller, and the small-diameter end becomes larger. The medicine stored in the insulin storage chamber 40 is extruded downward from the conical holes 31 to achieve the effect of vibration for administration. In addition, The piezoelectric ring vibrates radially after being connected to alternating current, and ultrasonic waves are induced by the vibration, the induced ultrasonic waves can promote the absorption of insulin by human body.

The sealing rings 60 are arranged between the film 30 and the upper housing 10 and between the piezoelectric ring 50 and the lower housing 20. The sealing rings 60 are O-shaped sealing ring, and can be made of rubber or silica gel, for example. The connection between the parts is closer through the sealing rings 60, the sealing performance is high, and avoiding the leakage of medicine.

A liquid inlet 11 is formed in the upper housing 10, and a rubber plug 12 is arranged at the liquid inlet 11. The rubber plug 12 is used to tightly plug the liquid inlet 11 to prevent impurities from entering the insulin storage chamber 40 and contaminating insulin. After the insulin in the insulin storage chamber 40 is used up, the rubber plug 12 can be removed to supply insulin into the insulin storage chamber 40 through the liquid inlet 11, so that the insulin injection pump can be continuously used for multiple times.

The present disclosure also provides a manufacturing method of an insulin injection pump, as shown in FIG. 7, including the following steps:
S1, preparing a film 30: taking a sheet with a thickness of 1-5 mm, and forming conical holes 31 by laser etching or ion selective etching therein;
S2, preparing a piezoelectric ring 50: selecting piezoelectric materials to prepare a ring with a same outer diameter as that of the film 30; or performing sputtering deposition of a layer of piezoelectric materials through mask holes at an outer edge of the film 30 to form the piezoelectric ring 50 which is integrated with the film 30;
S3, preparing an upper housing 10: integrally molding an upper housing through 3D printing or injection molding;
S4, preparing a lower housing 20: integrally molding a lower housing through 3D printing or injection molding; and
S5, assembling: sequentially placing a sealing ring 60, the film 30, the piezoelectric ring 50 and a sealing ring 60 in the upper housing to seal and connect the lower housing 20 with the upper housing 10.

Specifically, the film 30 is made of metal, rubber or elastomer. The piezoelectric ring 50 is made of piezoelectric materials, such as piezoelectric crystals, piezoelectric ceramics or piezoelectric polymers. The upper housing 10 and the lower housing 20 are integrally molded by metal or plastic material, and the upper housing 10 and the lower housing 20 can be sealed and connected together by clamping, threads and glue. The preparing sequence of the film 30, the piezoelectric ring 50, the upper housing 10, and the lower housing 20 is in a random order, and can be adjusted according to actual production situations.

In another preferred embodiment, sputtering deposition of a layer of piezoelectric materials is performed on the film 30 through mask holes to form the piezoelectric ring 50, so that the film 30 and the piezoelectric ring 50 are formed as a whole. The piezoelectric ring 50 vibrates after being energized, and the vibration can be effectively transmitted to the film 30 to, drive the film 30 to vibrate together, so that transmission loss is reduced. The preparing sequence of the film 30, the upper housing 10, and the lower housing 20 is in a random order and can be adjusted according to actual production situations. The piezoelectric ring 50 must be prepared after the preparing process of the film 30, and is directly integrally molded on the film 30.

### Embodiment II

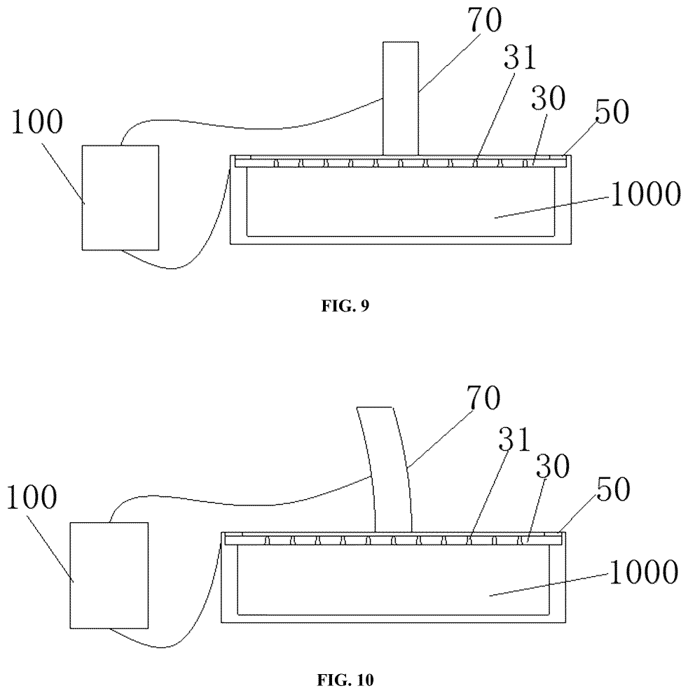

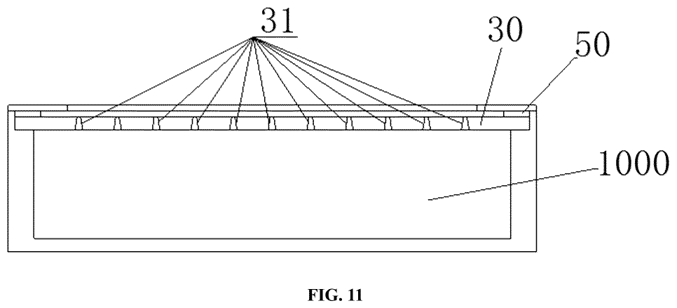

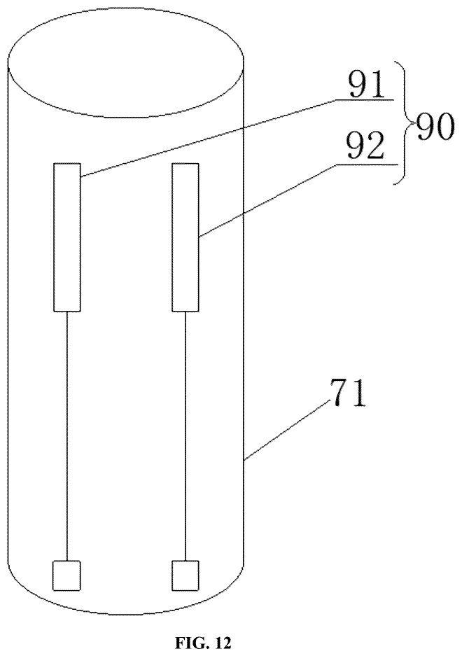

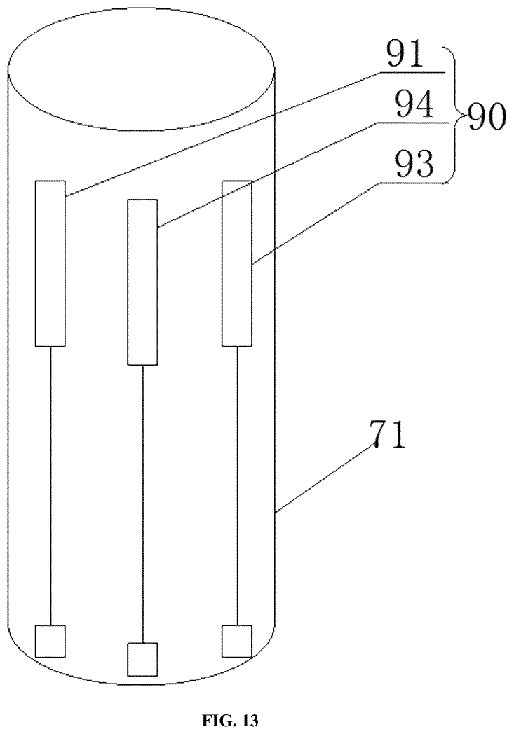

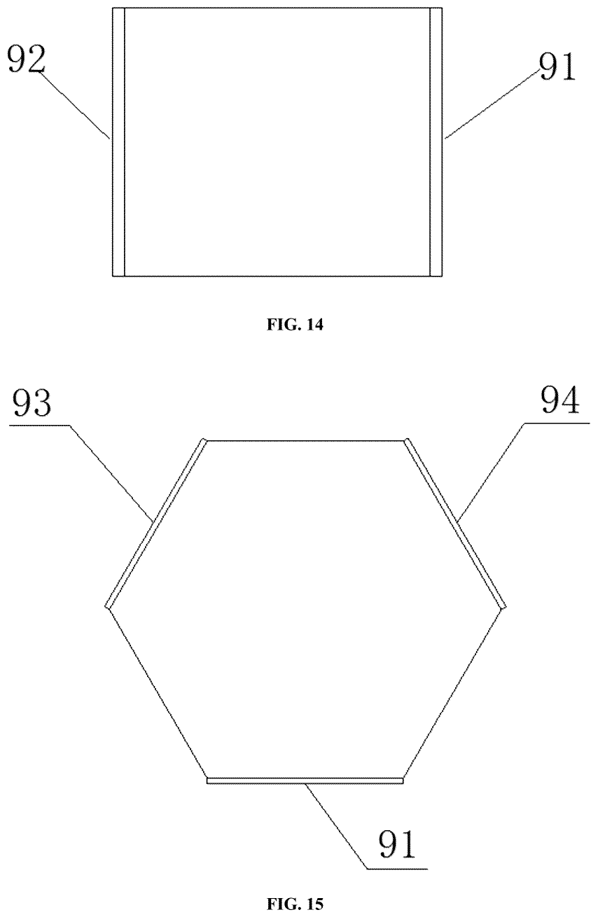

As shown in FIG. 8 to FIG. 15, the embodiment of the present disclosure discloses a closed-loop system. The closed-loop system includes an insulin injection pump 1000, a hose sensor 70, and a control unit 100.

The insulin injection pump 1000 includes a pump body. The pump body is used for storing insulin, and a film 30 is arranged in the pump body. The film 30 is made of hard film materials or flexible film materials, such as stainless steel, gold, copper, zinc, platinum, silver, tungsten, aluminum, aluminum alloy, natural rubber, isoprene rubber, polybutadiene rubber, styrene-butadiene rubber, nitrile rubber, chloroprene rubber, butyl rubber, halogenated butyl rubber, ethylene-propylene methylene rubber, ethylene propylene diene methylene rubber, epichlorohydrin rubber, polyacrylate rubber, silicone rubber, fluorosilicone rubber, fluororubber, chlorosulfonated polyethylene, hydrogenated nitrile rubber, thermoplastic polyolefin elastomer, thermoplastic styrene elastomer, polyurethane thermoplastic elastomer, polyester thermoplastic elastomer, polyamide thermoplastic elastomer, halogen-containing thermoplastic elastomer, ionic thermoplastic elastomer, ethylene copolymer thermoplastic elastomer, 1,2-polybutadiene thermoplastic elastomer, trans-polyisoprene thermoplastic elastomer, melt-processed thermoplastic elastomer, thermoplastic vulcanizate and polydimethylsiloxane.

Several conical holes 31 are formed in the film 30. The conical holes 31 are formed in the film 30 by laser etching or ion selective etching. Large-diameter ends of the conical holes 31 are located at a side adjacent to the insulin storage chamber, namely, the large-diameter ends of the conical holes 31 are liquid inlet ends, and the small-diameter ends of the conical holes 31 are liquid outlet ends.

A piezoelectric ring 50 is also arranged on the film 30. The piezoelectric ring 50 is arranged on a side of the small-diameter ends of the tapered holes 31. The piezoelectric ring 50 is made of piezoelectric materials, such as piezoelectric crystals, piezoelectric ceramics or piezoelectric polymers. During preparing, sputtering deposition is performed on a layer of piezoelectric materials through mask holes at the outer edge of the piezoelectric ring 50 to form the piezoelectric ring 50, so that the piezoelectric ring 50 and the film 30 are formed as a whole.

The film 30 and the piezoelectric ring 50 are connected with an external alternating current power supply through wires. Specifically, alternating current with the voltage of 10 V to 100 V is used for the external alternating current power supply. Also, an external direct current of 1 to 10 V can be used, and is converted into alternating current of 10 V to 100 V through a circuit.

During operation, the film 30 and the piezoelectric ring 50 are energized. The piezoelectric ring 50 vibrates after being connected to alternating current. The film 30 is driven to stretch or bend. The conical holes 31 continuously expand and contract under the stretching or bending action of the film 30, and the aperture thereof changes accordingly, so that insulin stored in the pump body is extruded.

The hose sensor 70 includes a tubular base 71. The tubular base 71 is generally tubular-shaped, and a chamber is formed in the longitudinal direction of the tubular base and can be used as an injection channel for insulin. Specifically, the hose sensor 70 is connected with the insulin injection pump 1000. A liquid inlet end of the tubular base 71 is engaged with a liquid outlet end of the pump body for injecting insulin from the insulin injection pump 1000 into human body through the hose sensor 70.

Electrodes 90 are arranged on an outer wall of the tubular base 71, and the electrodes 90 include a working electrode 91 and an active electrode 92. The active electrode 92 plays the role of circuit connection and voltage stabilization for the hose sensor 70. Reagent enzyme is arranged on the working electrode 91. When the glucose concentration of the patient is detected by the hose sensor 70, the reagent enzyme on the working electrode 91 reacts with the corresponding components in the body fluid of the patient to produce a product, so that an electrical signal is send by the working electrode 91. Specifically, fixed glucose oxidase, such as glutaraldehyde and chitosan, can be used as reagent enzyme. When glucose concentration in the subcutaneous interstitial fluid changes, glucose is catalyzed by glucose oxidase to produce hydrogen peroxide, and hydrogen peroxide is oxidized or reduced at the working electrode 91 to cause a change in the current, and then the sensor outputs an electrical signal.

In the embodiment, the working electrode 91 can be made of carbon paste, gold, platinum, carbon composite, gold composite or platinum composite. The active electrode 92 can be made of silver-silver chloride. The working electrode 91 and the active electrode 92 can be processed and formed on the tubular base 71 by processing methods, such as screen printing, ink-jet printing, micro-nano fabrication evaporation, or sputtering.

The electrodes 90 may include a working electrode 91, a counter electrode 93 and a reference electrode 94. At that time, the counter electrode 93 plays the role of circuit connection for the hose sensor 70, and the reference electrode 94 plays the role of voltage stabilization for the hose sensor 70. Specifically, the working electrode 91 can be made of carbon paste, gold, platinum, carbon composite, gold composite or platinum composite. The counter electrode 93 can be made of carbon paste, gold, platinum, carbon composite, gold composite or platinum composite. The reference electrode 94 can be made of silver-silver chloride. The working electrode 91, the counter electrode 93 and the reference electrode 94 can be processed and formed on the tubular base 71 by processing methods such as screen printing, ink-jet printing, micro-nanofabrication evaporation or sputtering.

The cross section of the tubular base 71 is circular or polygonal, wherein the polygon can be rectangular, regular pentagonal, regular hexagonal, for example. The tubular base 71 can be made of one of the following: polytetrafluoroethylene (PTFE), polyethylene terephthalate (PET), polyvinyl chloride (PVC), glass fiber (FR4), polyurethane fibroin, chitosan, polylactic acid, polycarbonate, polyurethane (PU), polyester, polypropylene (PP), polyethylene (PE), polyimide, polyimide thermoplastic polyurethane elastomer (TPU), silica gel, rubber, latex, thermoplastic elastomer (TPE), or perfluoroethylene propylene copolymer (FEP). The length of the tubular base 71 is 1 mm to 15 mm, such as 1 mm, 2 mm, 5 mm, 8 mm, 10 mm, 12 mm, and 15 mm, and the inner diameter of the base is 100 μm to 1000 μm, such as 100 μm, 200 μm, 300 μm, 500 m, and 1000 μm.

The control unit 100 includes a signal acquisition module 101, a control module 102, and an execution module 103. An input end of the signal acquisition module 101 is connected with an output end of the hose sensor 70. An output end of the signal acquisition module 101 is connected with an input end of the control module 102. The input end of the control module 102 is connected with an input end of the execution module 103. An output end of the execution module 103 is connected with an input end of the insulin injection pump 1000. The signal acquisition module 101 is used for receiving an electrical signal from the hose sensor 70 and converting the signal, and the electrical signal detected by the hose sensor 70 is transmitted to the control module 102. The control module 102 analyzes and judges the electrical signal collected by the signal acquisition module 101, and then sends instructions to the execution module 103. The execution module 103 controls the start and stop of the insulin injection pump 1000 according to the instructions sent by the control module 102.

### Embodiment III

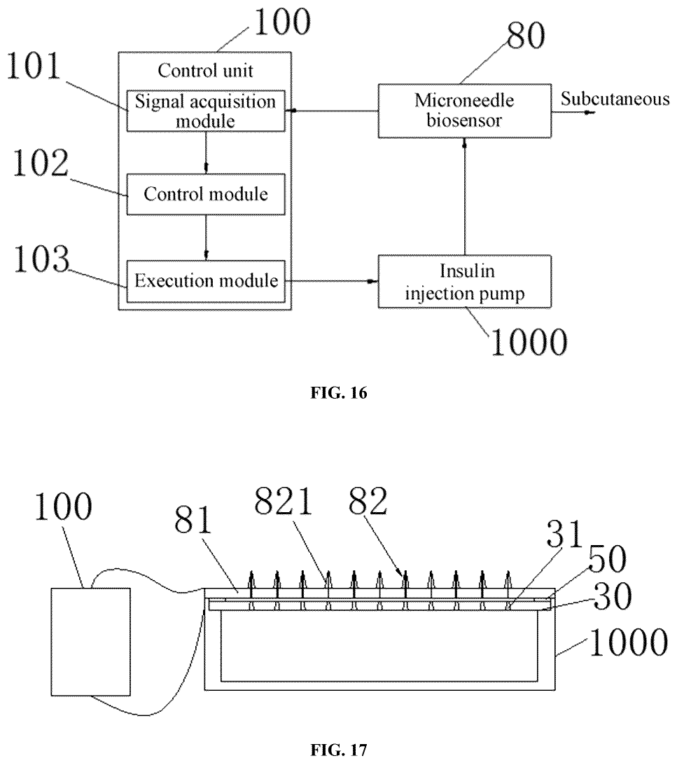

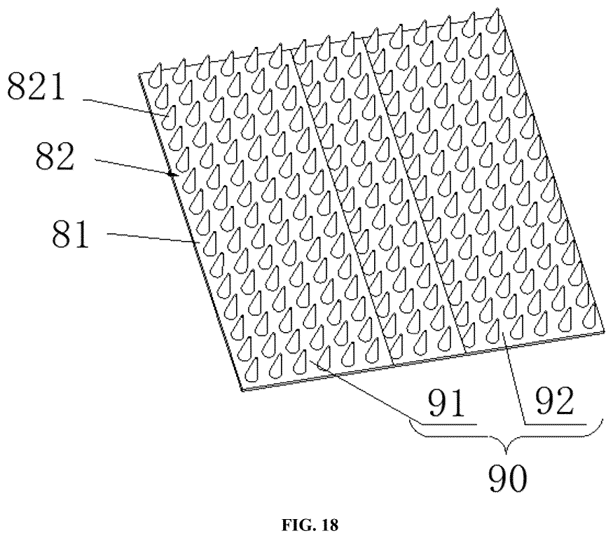

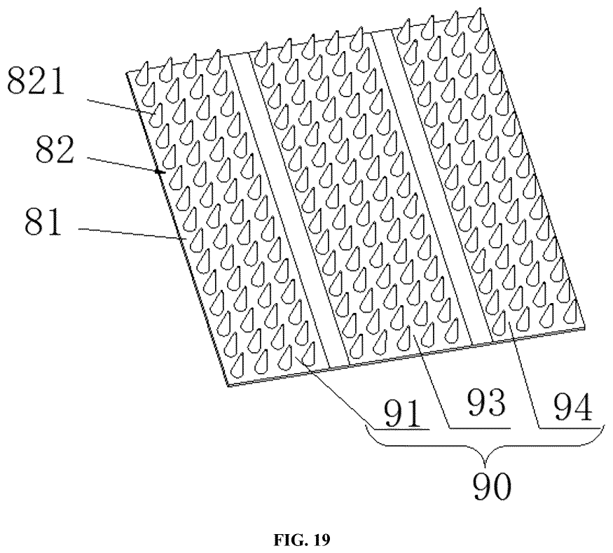

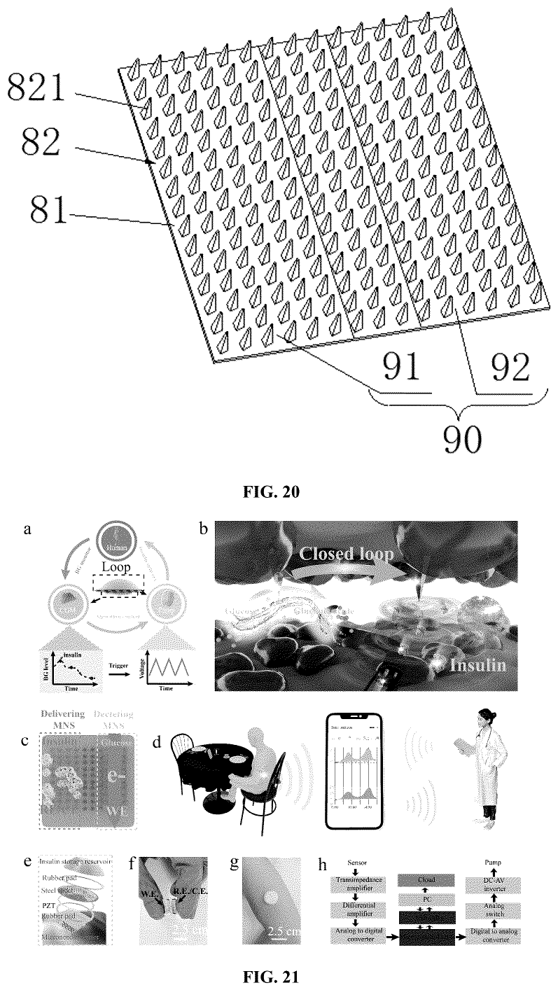

As shown in FIG. 16 to FIG. 20, the closed-loop system disclosed by the embodiment is further improved on the basis of the second embodiment. The sensor in the embodiment is a microneedle biosensor 80.

Specifically, the microneedle biosensor 80 includes a substrate 81, a microneedle array 82 and electrodes 90. The substrate 81 is integrally formed with the microneedle array 82. The microneedle array 82 includes several hollow microneedles 821 arranged in an array. The hollow channel in each hollow microneedle 821 is an insulin injection channel. Each hollow microneedle 821 is conical or pyramidal-shaped. The large-diameter end of each hollow microneedle 821 is connected with the substrate 81, and the small-diameter end of each hollow microneedle 821 forms a tip for penetrating into the skin. Specifically, the height of each hollow microneedle 821 may be 300 to 2000 μm, the diameter of each hollow microneedle 821 on the substrate 81 may be 50 to 1000 μm, and the thickness of the side wall of each hollow microneedle 821 may be 30 to 300 μm.

The substrate 81 is covered with the electrodes 90. The electrodes 90 include working electrodes 91 and active electrodes 92. The working electrodes 91 are made of nano-gold composite, and the active electrodes 92 are made of a nano-silver/silver chloride composite paste. During fabrication, the electrodes can be fabricated on the outer layer of the microneedle array 82 protruding from the substrate 81 or on the inner layer of the microneedle array 82 being recessed in the substrate 81. When the working electrodes 91 and the active electrodes 92 are fabricated on the outer layer of the microneedle array 82 protruding from the substrate 81, the working electrodes 91 and the active electrodes 92 do not penetrate into the inner layer of the hollow microneedle array 821. At this time, only the microneedles need to be in contact with a detected solution, and the detected solution does not need to flow into the hollow channels of the microneedle array 82. Meanwhile, each working electrode 91 includes an electrode layer, a Prussian blue layer, a reagent enzyme layer, and a biocompatible polymer layer laminated on the substrate 81, wherein the electrode layer can be made of gold, platinum or carbon. The biocompatible polymer layer is formed by covering a liquid biocompatible polymer on the reagent enzyme layer, and then the liquid biocompatible polymer is heated and dried, so that the biocompatible polymer layer is formed. The biocompatible polymer layer can be made of perfluorosulfonic acid, and the biocompatible polymer layer can avoid the damage to human body caused by the Prussian blue layer.

In this way, when the working electrodes 91 make contact with the detected solution, the reagent enzyme can react with a corresponding analyte in the detected solution, and a product is produced through a reagent enzyme reaction, and the product is oxidized or reduced at the working electrodes 91 to cause a change in electrical signal.

The control unit 100 includes a signal acquisition module 101, a control module 102, and an execution module 103. An input end of the signal acquisition module 101 is connected with an output end of the microneedle biosensor 80. An output end of the signal acquisition module 101 is connected with an input end of the control module 102. The input end of the control module 102 is connected with an input end of the execution module 103. An output end of the execution module 103 is connected with an input end of the insulin injection pump 1000. The signal acquisition module 101 transmits the electrical signal detected by the microneedle biosensor 80 to the control module 102. The control module 102 analyzes and judges the signal, and then sends instructions to the execution module 103. The execution module 103 controls the start and stop of the insulin injection pump 1000 according to the instructions sent by the control module 102.

The using principle of a closed-loop system in the embodiment is as follows. After the microneedle array 82 penetrates through the skin, an electrochemical reaction occurs on the working electrodes 91, and the change in the electrical signal is transmitted to the signal acquisition module 101. Then, the signal acquisition module 101 transmits the electrical signal to the control module 102. The control module 102 analyzes the glucose concentration in interstitial fluid to determine whether insulin needs to be injected. If the glucose concentration in interstitial fluid increases, the control module 102 sends an instruction of injecting insulin to the execution module 103. The execution module 103 instructs the insulin injection pump 1000 to be energized. The piezoelectric ceramic ring vibrates radially to drive the film to vibrates radially along with it, so that the conical holes in the film stretch and bent continuously. Insulin is continuously injected into the hollow microneedles on the microneedle biosensor 80, and then into the skin, so that the detection of blood glucose concentration of diabetic patients and the automatic delivery of insulin are realized. During use, manual intervention is not needed, the insulin injection pump is convenient to use, the functions of measurement and treatment are both realized, and the integration level is higher. Moreover, the insulin injection pump 1000 and the microneedle biosensor 80 are small in size, convenient to carry and wearable, and low in cost.

### Embodiment IV

### Preparation and Degradation of the Microneedles

A negative PDMS microneedle mold was purchased, followed by silicon oxidation. After treating with the PDMS mold in ozone cleaner for 5 min, the surface became hydrophilic. A 10 μl of 5% octyl trichlorosilane in benzene was heated at 60° C. for evaporation to modify the PDMS mold for 16 min. Then, a fresh PDMS liquid was poured into the modified PDMS mold, followed by heating at 80° C. for 2 h for curing. After peeling off, a positive microneedle mold was obtained. 2% polylactic acid in dichloromethane was poured onto the positive microneedle mold and dried, and after peeling off, the PLA hollow microneedles together with the PLA baseplate were obtained. To study the biodegradation, the PLA microneedles was placed in the buffer at pH 7.5 at room temperature, and every month it was taken out for drying. Then, the changes in weight were measured by an electronic precision balance.

### Preparation of the Sensing Electrodes

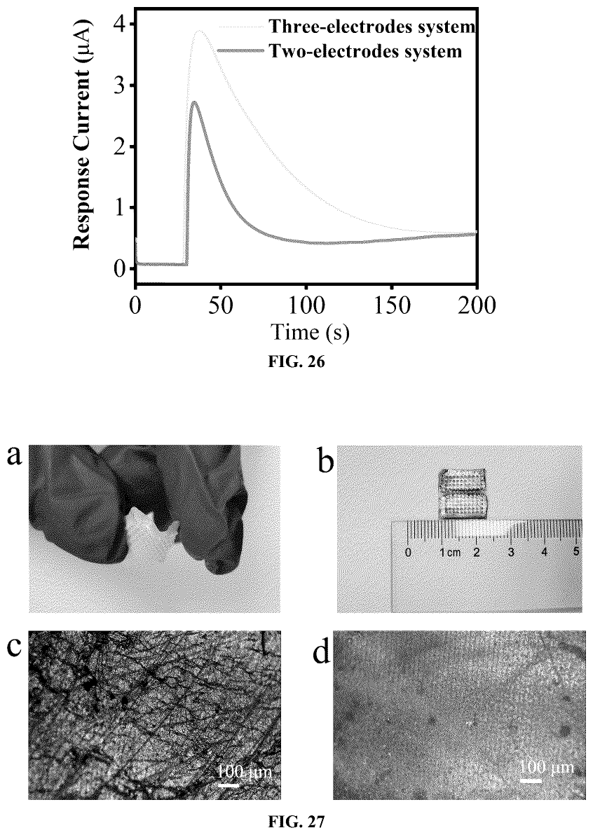

A two-electrode sensing system was used in this work, since the difference in electrochemical performance between two and three-electrode systems was not significant (FIG. 26, Supporting Information). Thin-film electrodes were obtained by sputtering. 5 nm Ti/200 nm Au was deposited onto the PLA microneedles in two areas to obtain two Au electrodes. One area was for the working electrode 91, and the other was for the reference electrode 94 or the counter electrode 93. A thin-film of Ag was deposited onto one Au electrode with a thickness of 200 nm. A 30 μl of 0.05 M FeCl3 solution was placed on the second area for 30 s to convert the Ag electrode to be Ag/AgCl electrode as the reference electrode 94 or the counter electrode 93, which would stabilize the working potential to further avoid the excessive drift of current baseline. To obtain a nanostructured Au (N—Au), an original Au (O—Au) was applied in buffer at a current at 0.015 A for 10 s using an electrochemical workstation in DC mode. The Au electrode was used as the working electrode 91 to deposit PB on it, with a commercial Ag/AgCl cylinder as a reference electrode 94 and a commercial Pt wire as a counter electrode 93. The impurities on the working electrode 91 surface were removed by cyclic voltammetry (CV) in H2SO4 at a 1 V/s scanning rate for 20 cycles. Then, the electrode was cleaned twice with 0.1 M KCl/0.1 M HCl solution. A 200 μl of PB solution (2.5 mM FeCl3, 2.5 mM K3Fe (CN)6, 0.1 M KCl/HCl) was added to the electrode surface and scanned by CV at a 20 mV/s scanning rate between −0.2 to 0.5 V for 2 cycles. Then, a new PB solution was added and scanned for 2 cycles again. After that, 0.1 M KCl/0.1 M HCl solution was added, and a CV scanning with a 50 mV/s rate was used twice at 0.2-0.5 V to stabilize PB. After electrodepositing PB on nanostructured gold, N—Au/PB (nanostructure gold/Prussian blue) was obtained. O—Au/PB was obtained after depositing PB on original Au without the electroetching.

To immobilize glucose oxidase (GOD), a 2% glutaraldehyde solution and a 10 U/μl GOD solution were mixed, and the volume ratio was 1:1. A 6 μl of the mixture was placed onto the working electrode 91. After drying, a 6 μl of 1% nafion was further deposited onto the enzyme layer. The sensor was dried overnight at 4° C.

### Electrochemical Characterization of the Sensor.

The sensing measurements were performed at room temperature (21° C.). The N—Au/PB electrode functioned as the working electrode 91, and the Ag/AgCl electrode functioned as the reference 94 electrode or the counter electrode 93. The EIS testing ranged from 1×10−2 to 1×104 Hz. The sensing responses for H₂O₂ and glucose were obtained from the currents at 100 s in a 50 mM buffer solution, with an applied working potential of −0.1 V versus Ag/AgCl.

To study the effects from the interferences, 5 μl of 2 mM lactate, 5 mM uric acid, 5 mM dopamine, 5 mM ascorbic acid, and 10 mM glucose were added sequentially to 200 μl buffer solution, and the potentiostat recorded the i-t curve.

To study the temperature effect, the sensor sample was placed on a hot plate, and the current feedback for glucose was studied in the temperature range of 20-40° C. To study the pH effect, The phosphoric acid solution with a pH 6-8 was used as a buffer for detecting glucose. To study the lifetime, the sensor was stored at room temperature for two weeks and was tested for the response current to glucose. To study the repeatability, adding and removing glucose in a buffer on the sensor were repeatedly for many times. To study reproducibility, 5 samples were used to test 20 mM glucose. To study the long-term stability of the sensor, the sensor was placed in the 20 mM glucose in the buffer for 10 hours. To study the effect of bending, a clamp was used to bend the sensor to different angles to detect glucose.

### The Preparation and Characterization of the Ultrasonic Pump.

A PZT ring and a SUS304 stainless steel with many conical holes 31 were integrated by using a glue. The resistivity of the SUS304 stainless steel sheet was 0.73 μΩ/m. It had a high elongation of 40% and a tensile strength ranging from 515 to 1035 MPa for resisting a high vibration intensity of the ultrasonic pump. The resonance impedance of the PZT ring was 180Ω, and its resonance frequency was 100 kHz with a direct capacitance of 3400 pF. The diameter of the stainless steel was 16 mm, and the thickness was 0.05 mm. The outer ring diameter of PZT was 15 mm, the inner ring diameter was 8 mm where the vibrating area occurred, and its thickness was 0.65 mm. The vibration area of the sheet was located in the inner ring, and its diameter was also 8 mm. The 900 conical holes 31 fabricated by laser cutting were located in the center of the stainless-steel sheet, and the conical hole area was a 4 mm circle. The diameter of the bottom conical hole was about 5˜6 μm and that of top was about 50 μm.

Then, the conducting wires was welded to the surfaces of the PZT ring and the steel sheet by a welding gun, respectively. A PCB for with two circuits was used to power the closed-loop system, and it was driven by a USB-cable that was connected to a computer by supplying a DC voltage of 5 V. The first circuit in the PCB processed the input DC voltage and converted it to an output DC voltage between 2.5 V to 5 V that can be set by the user. This voltage was the input voltage for the second circuit as a DC-to-AC converter to convert it to an AC voltage with a peak voltage about 100 V and a frequent of 100 kHz. The ultrasonic pump was driven by the high AC voltage for injecting insulin. To obtain different flow rates of the ultrasonic pump, different sets of DC voltages were applied at the DC-to-AC converter to drive the ultrasonic pump. In the characterization of the ultrasonic pump, the PCB at different powers to push insulin with various concentration, and the time durations required to drive 1 ml insulin were recorded.

The impedances at various frequencies were detected by an impedance analyzer at 0-1 MHz. The working current of the PCB driving the ultrasonic insulin pump was measured by an oscilloscope.

Study of Insulin Diffusion with Ultrasound.

The insulin-conjugated fluorescence microspheres were synthesized in-house using carbodiimide chemistry. The insulin-conjugated fluorescence microspheres were synthesized with a carbodiimide chemistry. Briefly, a 200 μl of carboxylate-modified microspheres (λ=605 nm) were washed and activated in a MES buffer (0.05 M, pH 6), followed by the addition of 5 mg EDC and 15 mg NHS. The unreacted reagents were removed by a centrifugation at 12000 rpm for 10 min. The supernatant was resuspended in an 800 μl of MES buffer (0.05 M, pH 7) to couple with a 100 μl diluted insulin. The mixture was incubated on a vortex shaker at room temperature for 2 h. After a further centrifugation, the microspheres were blocked by a blocking solution containing 1% BSA and 0.04 M ethanolamine for 30 min. The insulin-microspheres conjugates were washed for 3 times and dispersed in a 200 μl of tris buffer (20 mM, pH 8.0) containing 1% BSA and 0.1% surfactant S9. The final product was stored at 4° C. before use.

To study the effect of ultrasound, 2% agarose hydrogel was used to stimulate interstitial fluid for the insulin delivery. The PDMS worked as an artificial skin to cover the agarose hydrogel. A 5 μl of 10 U/μl insulin conjugated fluorescence microspheres were injected into the agarose hydrogel through the artificial skin by using a syringe. At the beginning, insulin was clustered in the injection area. Then, the microneedles with ultrasound and without ultrasound were placed on the artificial skin, and finally the fluorescence photos were taken by a microscope at different times.

### Integration of the Closed-Loop System.

A biocompatible stainless steel and a PZT ring contacted each other closely, and was integrated together by a waterproof glue as the ultrasonic pump, which was further placed between two rubber layers for absorbing the vibration. One side of the stainless-steel sheet contacted the PZT ring, and the other side of the stainless-steel contacted insulin. Thus, insulin would not touch the PZT ring. The insulin reservoir was fabricated by a 3D printer with ABS material that is biocompatible and non-toxic. The reservoir was further placed on top of the stainless steel and the rubber layer, and fixed by a waterproof glue. Finally, the back of the microneedle sensor was connected to the center part of the other side of the ultrasound pump without touching the edge where the PZT ring was located. The contact areas between each part were connected by a waterproof glue. The waterproof glue would not be damaged by ultrasound. Although ultrasound vibrate intensely, the glue can cushion the effect of vibration and would not be damaged.

### Induced Diabetic Rats.

Male SD rats weighing 140 g were reared at 24° C.±1° C. and 55%±5% humidity 5 days before the experiment. The light-dark cycle lasted for 12 h (lights on at 8:00 and lights off at 20:00). Rats were allowed to access to food and water freely. In the experiment, sodium citrate buffer (50 mM, pH 5.4) was prepared, and then a 1 ml of buffer was put into the centrifuge tube covered with aluminum foil. Before the injection, streptozotocin (STZ) was dissolved in the citrate buffer to prepare a 6 mg/ml STZ solution. Since STZ was degraded quickly in citrate buffer in 15-20 min, a fresh STZ solution must be prepared and injected within 5 min. A 40 mg/kg STZ solution was injected intraperitoneally with a syringe at room temperature. Then, the diabetic rats were provided with standard food and 10% sucrose water. For the next 4 days, the same amount of STZ solution and 10% sucrose water were injected daily. After 6 days, 10% sucrose water was changed into normal water, and diabetes was induced in rats after 9 days.

### Rats Experiments.

The study was approved by the Ethics Committee of Peking University First Hospital (approval number: 202061). The diabetic rats were selected as the experimental objects, and their blood glucose levels were about 20 mM, and a glucose level of 8.3 mM was determined to be the critical value for diabetic rats, which is the blood glucose standard value of rats considered to be suitable for the treatment of diabetes.24 Before the experiment, the animal anaesthetic isoflurane was poured into the evaporator, and the air pump was triggered. In the first anaesthesia stage, the rats were put into the anaesthesia glass box, and the air pump intensity was adjusted to 2 for 3.5 min. After the rats entered the anaesthesia state, they were taken out and placed on a fixed plate, and the head was connected to an anaesthesia mask, and then the limbs were fixed on the plate. In the second anaesthesia stage, the air pump intensity was maintained at 0.4 during the later experiment. After that, the rats' buttocks were shaved with a blade and soapy water, and the excess soapy water was removed with absorbent paper. Then, the closed-loop system was fixed at the shaved buttocks by the waterproof paste. The microneedles penetrated the skin, and the device was connected to a PCB and a computer with the conducting wires.

### Embodiment V

### The Design and Principle of the Close-Loop System.

FIG. 21(a) shows an overall working principle for a closed-loop mini-system in diabetes management. There are three components in a closed-loop system: 1) a microneedle glucose sensor for detecting blood glucose; 2) an ultrasonic pump based on a PZT and a steel sheet for delivering insulin; 3) a PCB with an algorithm based on a suspension control. The sensor monitors the blood glucose level continuously, when it senses the blood glucose level higher than the normal range, it would trigger the pump to inject insulin into the human body. They are fully integrated as a highly promising platform for artificial pancreas due to a wearable, minimally invasive and miniature nature.

FIG. 21(b) shows the schematic illustrations of the working mechanism of the ultrasonic pump-enabled closed-loop system. The microneedles were employed to penetrate the stratum corneum to enter interstitial fluid without pain and bleeding, and the microneedles served as the building blocks for both sensing glucose and delivering insulin. The microneedles consisted of two components: the sensing microneedles and the delivering microneedles, as shown in FIG. 21(c). The sensing microneedles served as the working electrode 91 for CGM to detect blood glucose. On the other hand, the delivering microneedles worked the channels to deliver insulin subcutaneously by the ultrasonic insulin pump and also were used as the reference electrode 94 or the counter electrode 93 for CGM. This design of the microneedles that separates the detection and the delivery can avoid the influences of the insulin injection on detecting glucose, such as the noise signal caused by the injecting flow and the instability of the gradient of glucose concentration because of the local insulin.

When the microneedles penetrated the skin to enter interstitial fluid, the circuit powered the sensing device to measure the current signal from interstitial glucose and further converted it to a predicted blood glucose level with the microprocessor to control turn on and off the insulin pump. If the blood glucose level was higher than the threshold value, the microprocessor triggered the ultrasonic pump to release insulin. Although there was some time lag (usually about 4-10 min) between blood glucose and interstitial glucose, some studies demonstrated that they were well correlated.25, 26 As shown in FIG. 21(d), the significance of this work is that it can be used as an artificial pancreas to help diabetes humans automatically regulate blood glucose balance during the meal or other activities. Also, it can transmit the data of the daily blood glucose levels and the amount of delivered insulin to a mobile APP, and accurately track and recode the fluctuations of glucose levels. What is more, the date can be sent to a health database cloud storage, which can formulate a reasonable therapeutic schedule and provide a dietary management proposal for doctors or patients themselves.

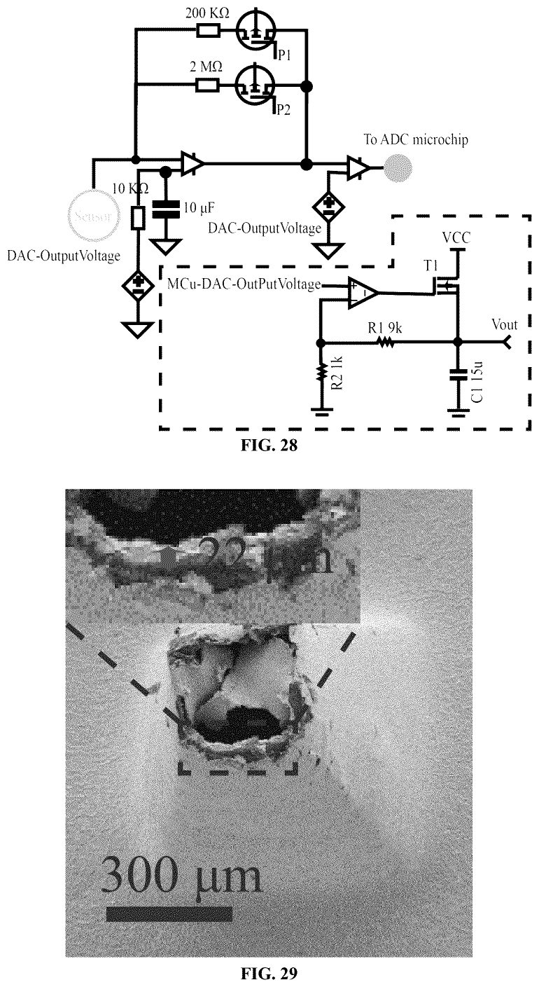

As shown in FIG. 21(e), the components of an entire device consisted of an insulin reservoir, an ultrasonic pump, and a microneedle array 82. The hollow microneedles 821 were in a 10×10 array with about 1 mm in height (FIG. 21(f)). Half of the microneedles worked as the working electrode (W.E.), and the other microneedles served as the counter electrode 93 or the reference electrode 94 (C.E/R.E.) (FIG. 27, Supporting Information). FIG. 21(g) illustrated that the device was 2.5 cm in diameter and 1 cm in thickness. It was much smaller than a commercial closed-loop system, and the small size was vital to users in terms of comfort and safety for wearing. As can be seen in FIG. 21(h), the closed-loop system consisted of a device and a PCB with software. The PCB circuit as detailed in FIG. 28 (Supporting Information) included a two-electrode constant-potential circuit, a signal conditioning circuit, a USB-cable power circuit, and a MCU and DC-AV inverter. The two-electrode circuit provided a stable potential for the chronoamperometric measurement, followed by the signal conditioning circuit to process the sensor current. Then, the analog signal was converted into the digital signal by a digital-to-analog port, and finally, the data was output to a PC and software with a USB cable. If hyperglycemia was sensed, a DC signal would be applied to a DC-AV inverter to trigger the ultrasonic insulin pump.

### Embodiment VI

### The Construction of the Microneedle Sensing Electrodes.

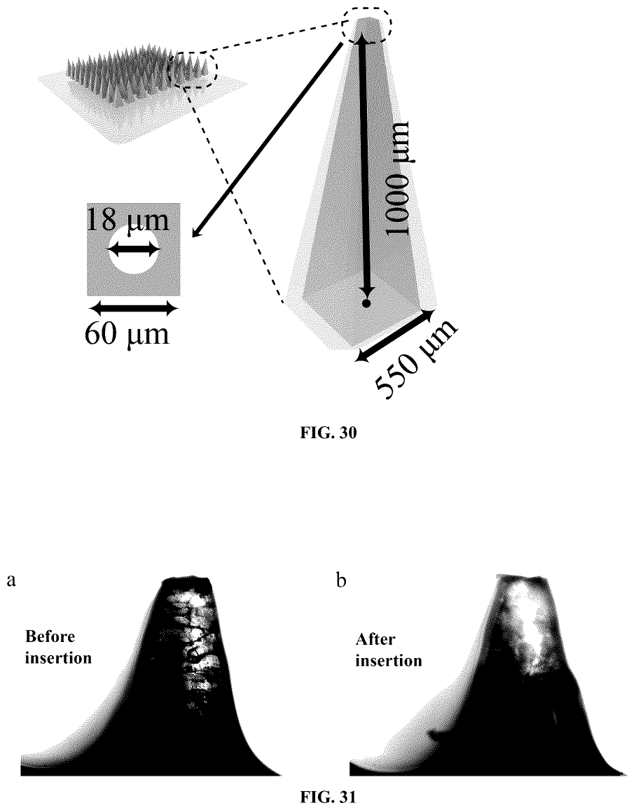

FIG. 22 shows the preparation and characterization of the sensing electrodes. FIG. 22(a) shows the fabrication process of the microneedles and the sensing electrodes. The injection molding method was used to prepare the microneedles with a low cost and a mass production. The microneedle array 82 in PDMS as a positive mold was obtained via soft lithography from a purchased PDMS negative mold. PLA is a polymer polymerized from lactic acid, and it has good biocompatibility, good degradability, outstanding transparency, and excellent tensile strength and ductility.27, 28 PLA was then cast onto the PDMS microneedles. After drying, the PLA layer was peeled from the PDMS mold to obtain the hollow PLA microneedles. Since the mold is reusable, this entire process for producing hollow microneedles 821 production was convenient and repeatable. To open the tips of the hollow microneedles 821, the volume of the PLA solution covered onto the PDMS mold was controlled carefully to reveal the tips of the mold, thus creating the pinhole of the microneedles. FIG. 22(b) shows the SEM images of the PLA microneedle arrays with approximately 1000 μm in height and 550 μm in square base size. The wall thickness of a microneedle was about 22 μm (FIG. 28, FIG. 30 and Table 1, Supporting Information). Through the force analysis, the results show that the PLA microneedle can penetrate the skin without breaking at this thickness of wall (FIG. 31 and microneedle force analysis in Supporting Information). The inset SEM image in the upper left of FIG. 22(b) shows the backside of the microneedles with the base plate made of PLA. However, due to the gravity effect, more PLA accumulated at the bottom of the microneedles, and at a higher height of a microneedle, the wall thickness was smaller. To analyze the biosafety of the microneedles when broken in skin, the degradability experiment of the PLA microneedles was performed by placing the microneedle arrays 82 in a buffer at pH 7.5. The result showed that half of the weight degraded within 120 days as shown in FIG. 22(c), demonstrating that microneedles are biodegradable when broken in skin.

TABLE 1
Dimensions of microneedle devices and individual needle

| Size of the | Number | Height | Size of | Size of | Thick- | Diameter |
|---|---|---|---|---|---|---|
| whole | of | of | needle | square | ness | of hole |
| microneedles | needles | needle | top | base | of wall | at the top |
| 15 × 15 × | 10 × 10 | 1 mm | 60 × | 550 × | 22 μm | 18 μm |
| 1 mm3 | | | 60 μm2 | 550 μm2 | | |

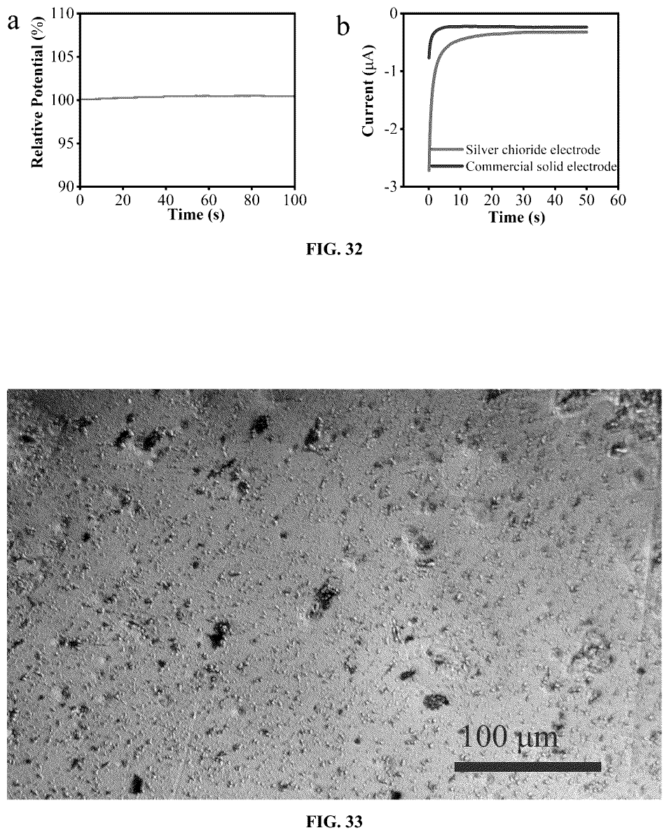

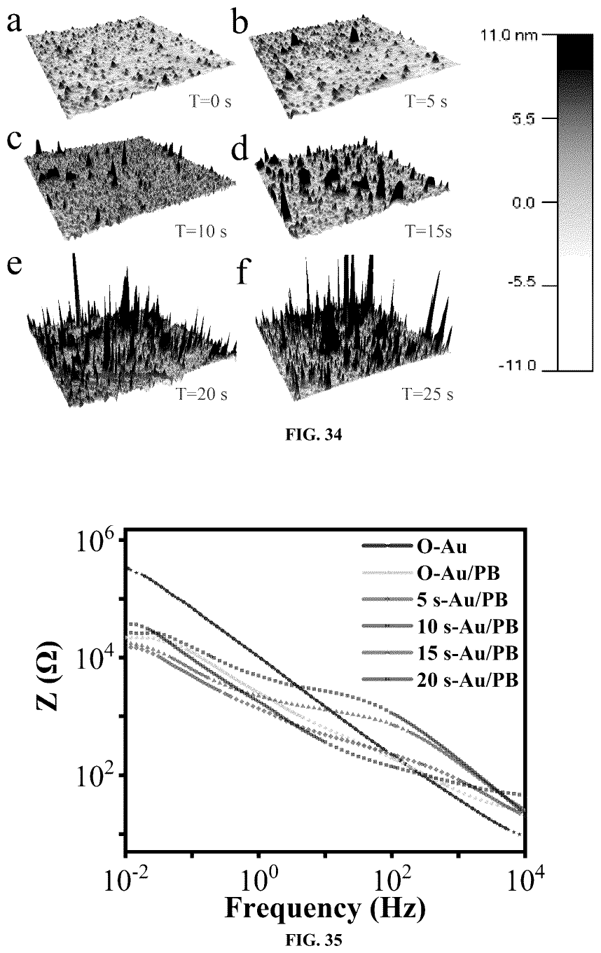

Further, two Au electrodes were fabricated onto the surface of the microneedles by sputtering, and Ag was further deposited on the surface of one Au electrode. The Ag electrode was then chlorinated to become Ag/AgCl as the reference electrode 94 and the counter electrode 93 of the sensor, providing a stable potential for sensing (FIG. 32, Supporting Information). Prussian blue (PB) is a strong catalyst for H2O2 at a low potential, and it can also increase the sensitivity and selectivity to reduce the influence of the interferences. Moreover, PB can increase the linear detection range of the sensor. However, its low thermal stability and pH stability are challenges for sensor stability. To improve the stability of PB, a high DC current electrolyzing H2O was applied at the Au-coating working electrode to produce a large amount of 02 on the surface. FIG. 22(d) illustrated that the injection flow caused by the collapse of 02 bubbles generated from the electrolysis of H2O can scour away part of Au, obtaining the sand-dune nanostructure with many holes on the Au surface, which increased the area for adhering PB, as shown in FIG. 21(e) and FIG. 33 (Supporting Information). As can be seen in FIG. 22(f), the AFM image of the Au electrode after being electrolyzed further showed that the sand-dune nanostructure was produced on the surface compared with the original Au, and more AFM images with various electrolyzing times were presented in FIG. 34 (Supporting Information). The sand-dune nanostructure would increase the attached surface area and enhance the adhesion to stabilize PB.

Then PB layer was electrodeposited on the Au electrodes as the working electrode 91 of the sensor, and the infrared image illustrated that the peak intensity of N—Au/PB was greater than that of original-gold/Prussian blue (O—Au/PB) at the vibration peak of C—N at 2140 cm-1. This indicated that PB had been deposited on the Au surface and the etched surface increases the contact area between PB and the electrode to result in more PB being deposited on the surface (FIG. 22(j)). After coating glucose oxidase (GOD), a layer of nafion was coated to enhance the biocompatibility of the electrode and block the interferences, such as ascorbic acid and uric acid. In addition, nafion further enlarged the linear range of the sensor by increasing the diffusion thickness of glucose.

Au was electrolyzed at a DC current of 150 mA for 5 s to 20 s to obtain different roughness on the surface, and a more roughness was obtained with an increasing electrolysis time (FIG. 22(g)), which provided a larger contacting area for the deposition and stabilization of PB. Further, with an increase of the electrolysis time, the peak of the CV current became bigger FIG. 22(h)), which meant more PB was deposited on the Au electrode due to the fractured surface. When the electrolysis time was 10 s, the current of the N/Au was about three times that of O—Au. However, as the further increase of the electrolysis time, the conductivity of the Au electrode became worse, resulting in a slower electron transfer rate and a smaller deposition amount.

The impedance semicircle radius of N—Au/PB electrode was substantially small compared to that of O—Au and O—Au/PB electrodes FIG. 22(i)). Since the electrode reaction kinetics controls the semicircle diameter, it demonstrates that the charge transfer impedance of the N—Au/PB was smaller than that of O—Au/PB. The reason is that an increased surface area of the N—Au film can enhance the electron exchange rate. Also, the slope of the low-frequency straight line from the N—Au/PB film is less than that of O—Au/PB (FIG. 22(i)). The impedance of N—Au/PB was much less than that of O—Au/PB (FIG. 35, Supporting Information). The detection of H2O2 would be more sensitive with a higher amount of PB, and the sensitivity from the N—Au/PB film was two times higher than that of the O—Au/PB film (FIG. 22(k)).

### Embodiment VII

### In-Vitro Sensing of Glucose.

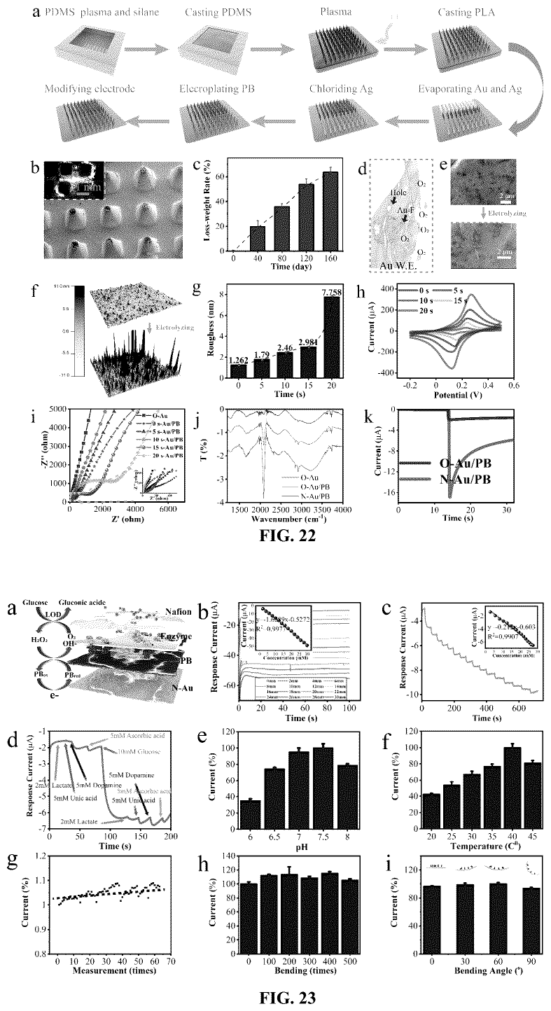

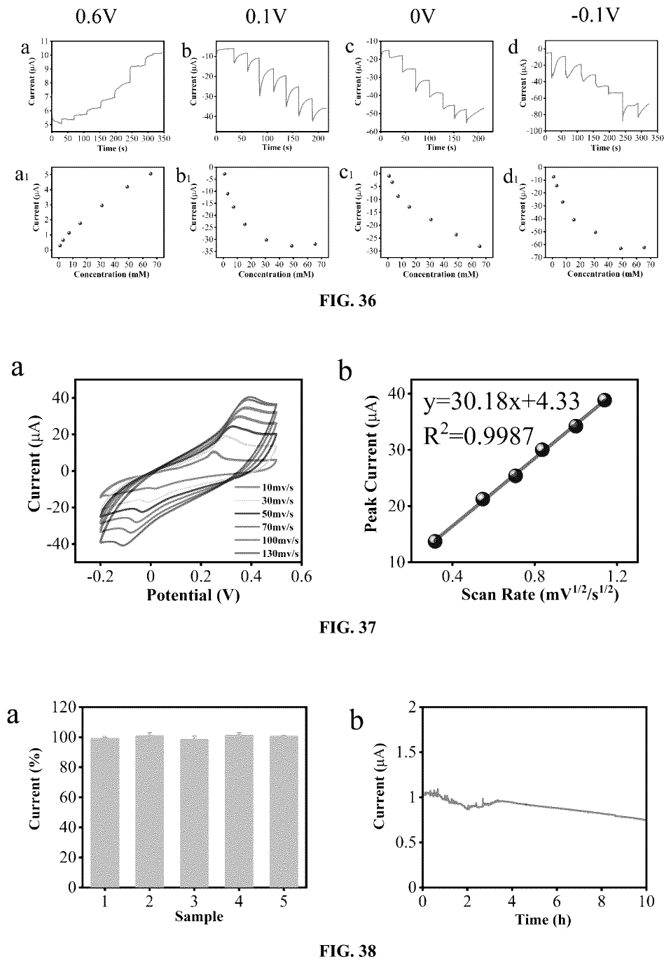

FIG. 23 shows the electrochemical characterization of the microneedle sensor for the in-vitro sensing glucose. The Ag/AgCl was the reference electrode 94 or the counter electrode 93, and the N—Au/PB electrode was the working electrode 91 at a potential of −0.1 V versus Ag/AgCl, which was used due to its higher sensitivity compared with other potentials (FIG. 36, Supporting Information).

As illustrated in FIG. 23(a), the structures from the top to bottom on the working electrodes 91 consisted of: 1) a nafion layer (it was biocompatible with the body), a GOD layer (a selective enzyme for glucose), a PB film (the oxidizing agent between H2O2 and free electron at low potential), and the W.E. Au (the inert electron conductor). With the presence of GOD, the glucose molecules were oxidized to generate H2O2, followed by being mediated via PB to produce a current response that can determine the glucose concentration. FIG. 23(b) showed the current response baselines being measured chronoamperometrically for various concentrations of H2O2, indicating an excellent sensitivity (1.6 μA/mM) and a linear relationship (R2=0.997), which is vital for detecting a low concentration of interstitial glucose. FIG. 23(c) shows the current response curve produced by placing a buffer droplet at a 0-27 mM glucose solution. The sensitivity for glucose was 0.212 μA/mM, and the correlation is 0.991, indicating that the sensor has a high electrochemical performance for detecting glucose.

To analyze the glucose reaction on the microneedle sensor, the cyclic voltammetry was performed on the W.E. electrode at 20 mM glucose buffer solution. The result of CV curves at different scan rates showed the peak current was linearly correlated to the square roots of the scan rate, and this demonstrated the reacting process was controlled by the diffusion rate in accordance with the Randles-Sevcik equation (FIG. 37 Supporting Information).

The selectivity of the sensor is essential for detecting glucose, since the metabolites (such as creatinine, uric acid, lactic acid, and ascorbic acid) in interstitial fluid may cause some errors in the accuracy of the sensor. Therefore, a detection potential of −0.1 V versus Ag/AgCl was set to avoid these non-target metabolites endowing the free electrons. What's more, coverage of nafion on the working electrode 91 was employed to repel these non-target metabolites. FIG. 23(d) demonstrates that the current responses to various interferences on sensing could be neglected compared with that of glucose, and the current fluctuation of non-target metabolites may be caused by the physical-mechanical movement of the solution when dropping the metabolites.

FIGS. 23(e) and (f) show the influences of pHs and temperatures on sensing, respectively. The pH of interstitial fluid in the human body is pH 7.3-7.4, and it shows that the sensing current for glucose was unchanged in the range of pH 7.0-7.5 (FIG. 23(e)), while for other pH ranges, the influences are significant. An extreme pH affects the hydrogen bonds and salt bridges of the enzyme, resulting in a decrease in current as the residues of the enzyme are no longer used for functional or structural stabilization. Likewise, the three-dimensional folded structure of the enzyme expands at a high temperature, leading to inactivation. Thus, the maximum sensing current occurs at 40° C. FIG. 23(f) shows that within 36 to 40° C. of human temperature, the change in current response was about 10%, and such change was still acceptable. These results indicate that the sensor could have excellent pH and temperature stabilities for practical applications on humans. The study of repeatability indicated that the sensitivity of the sensor was consistent during 60 consecutive measurements FIG. 23(g)), and the sensitivity varied within 5% for different samples (FIG. 38(a), Supporting Information). The long-term stability was investigated using a constant glucose solution, and the results showed that the current decreased by 25% within 10 hours, mainly due to the depletion of glucose in the solution (FIG. 38(b), Supporting Information). The flexibility of the device is vital because the microneedle sensor needs to withstand the wearing and bending when being worn on the body. The mechanical deformations were performed to test the flexibility of the sensor with repeated bending.

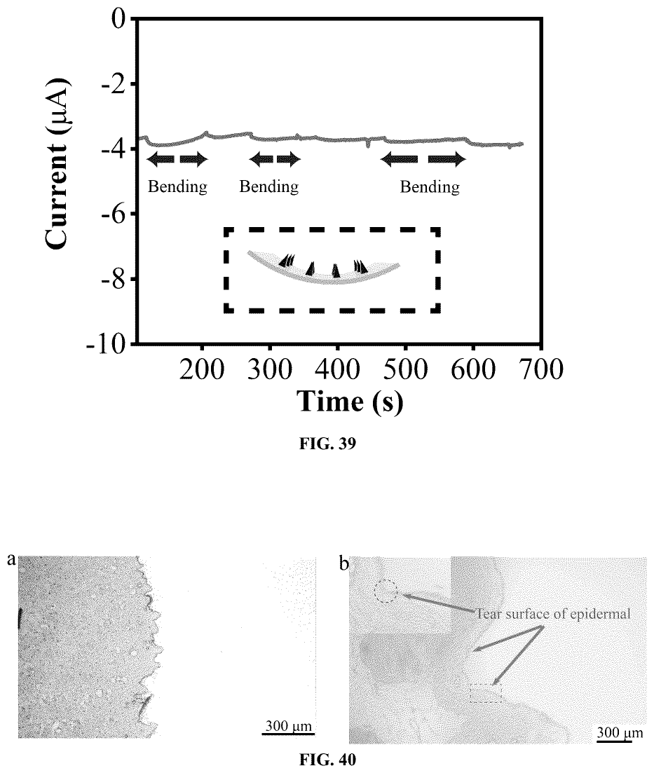

FIG. 23(h) demonstrates that the sensor displayed a high stability over hundreds of times of bending. For example, after 500 times bending cycles, the sensor still maintained about 100% of the initial sensitivity (FIG. 23(g)). FIG. 21(a) (Supporting Information) shows an image of the bent PLA microneedles to demonstrate the excellent flexibility of the sensor. The bending angles had a negligible influence on the sensitivity, as can be seen in FIG. 23(i). After bending at 60°, the current of the sensor changed by only about 5% and returned to the original level rapidly after stopping bending FIG. 39, Supporting Information). The results demonstrate that bending did not reduce the performance of the sensing electrode.

### Embodiment VIII

### Principle and Performance Characterization of the Ultrasonic Pump.

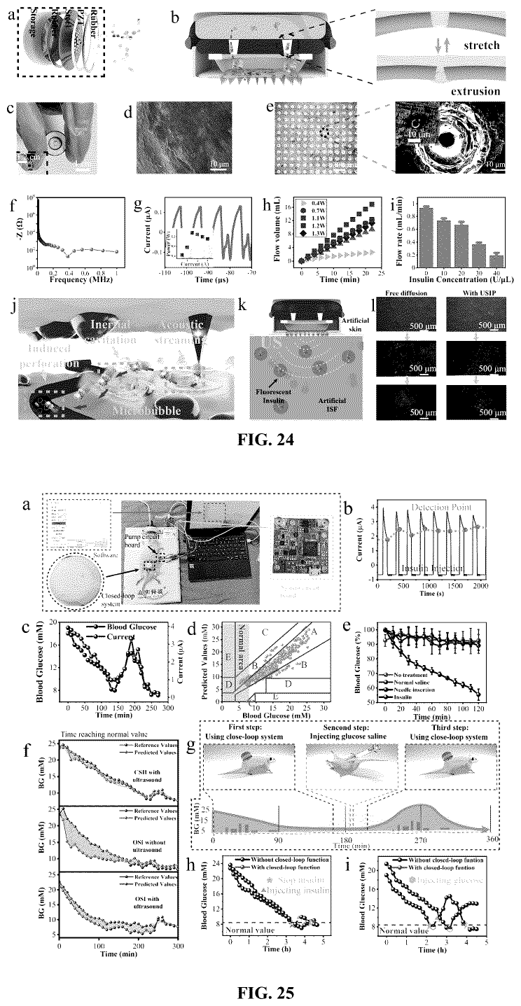

According to the instruction for use of rapid-type as part insulin product for human, the daily amount of insulin for human is 1 U/kg, whereas a diabetic rat is approximately 0.4 kg. Therefore, the required daily amounts of insulin are approximately 0.4 U/day for a diabetic rat, and 50 U/day for a human being. Considering that the model rats have much higher blood glucose (about 20 mM) than most human diabetes patients (about 10 mM), a higher amount of insulin than 0.4 U/day may be required. In addition, type 2 diabetic rats were used in this study that can develop insulin resistance, and this would lower the effect of insulin. Further, insulin delivered by the microneedles may leak out of the body during the injection into the dermal layer, and more insulin can be required. Some studies showed that more insulin was needed for injecting into diabetic rats as well. For example, Li et al. injected 50 U insulin subcutaneously into STZ rats to achieve a glucose-lowering rate of 6.5 mM/h, and this injected amount of insulin was much larger than the theoretical value (0.4 U).20 Therefore, it would be desired to have a micropump with a large driving force to provide adequate insulin injections. A PZT-based device can provide a large driving force. The devices that combine PZT and stainless steel are commonly used as atomizers. Rather than atomization, its microporous telescoping structure that allows for electrically controlled generation of driving force on the film size is studied for the first time in this work to generate continuous driving force in a micropump system. Meanwhile, some studies have shown that the use of PZT vibrating stainless steel membranes to generate ultrasonic fields can further accelerate the diffusion of drugs. This work proposes the ultrasonic pump to deliver insulin subcutaneously for the first time. When an interstitial glucose level exceeding the threshold value is monitored, the control algorithm triggers the ultrasonic insulin pump to deliver insulin into the dermis layer through the hollow microneedle channels. FIG. 24(a) exhibits the structure of the ultrasonic insulin pump. The main components are a PZT ring and a stainless-steel sheet with hundreds of conical holes 31, and the steel sheet is attached to the PZT ring. The piezoelectric effect from the PZT ring drives the steel sheet to function as a pump. Once an AC current is applied, the PZT ring vibrates radially to trigger the radial movement of the steel sheet, and the conical holes 31 in the steel sheet can be extended and bent repeatedly, to further generate a downward flow of insulin in the conical holes 31 (FIG. 24(b)). When being bent, insulin was pushed from the conical holes 31 because of the reducing volume of the hole space. When being extended, the conical hole 31 created an enclosed space as a valve to prevent insulin from flowing back to the drug reservoir. FIG. 24(c) displays the photograph of an ultrasonic insulin pump with a PZT ring and a steel sheet, and the diameter of an ultrasonic insulin pump is 2 cm with a thickness of 2 mm, and the SEM image of PZT can be seen in FIG. 24(d). As illustrated in the SEM image of the micro-pore of the steel sheet (FIG. 24(e)), hundreds of conical holes 31 were formed in the center of the sheet by laser cutting. The diameter at the top of each hole was about 50 μm, and the bottom is 5 μm.

The resonant frequency of the PZT ring is 100 kHz measured by an impedance analyzer (FIG. 24(f)), and a high frequency can provide an obvious insulin flow. In FIG. 24(g), the current wave of the PZT with an applied AC voltage was detected by an oscilloscope, and the powers at different currents were calculated. FIG. 24(h) shows the insulin flow rates driven by the ultrasonic pump at various powers, the pump can achieve high flow rates for delivering insulin, and the maximum flow rate can be 0.8 ml/min at 1.1 W. FIG. 24(i) shows that the flow rate decreased with an increased insulin concentration that hinders the movement of the liquid.

Some studies have demonstrated that high-amplitude sound waves propagated through interstitial fluid can spontaneously nucleate and excite microbubbles. The strong collapsed microbubbles, emit shock waves and generate directional jets in the fluid to move the drug molecules, which is called cavitation. The related physical effects of this inertial cavitation include a micro-flow and a fluid injection, and when interacting with cells or tissues, these would induce a reversible perforation at the plasma membrane, open the endothelial connections, and stimulate an endocytosis to overcome the diffusion resistances of the vessel wall and the plasma membrane (FIG. 24(j)). In our work, owing to the integration of the sensing microneedles and the delivering microneedles in the same area, the excessive insulin around the microneedles would decrease the amount of the surrounding glucose molecules, and this would cause a poor correlation between the blood glucose level and the local interstitial glucose level. Thus, ultrasound was introduced into the system to accelerate the diffusion of insulin and improve the accuracy of the sensor.

To evaluate the effect of ultrasound on enhancing the diffusion of insulin, a fluorescent characterization was performed in a simulated condition. The insulin-conjugated fluorescent microspheres were injected into a simulated interstitial fluid of agarose, and an artificial skin made of PDMS was covered on the simulated interstitial fluid. Then, a microneedle device with an ultrasonic insulin pump was inserted into the artificial skin, as shown in FIG. 24(k). As can be seen in FIG. 24(l), the fluorescence photographs illustrated that the fluorescence dissipated faster by ultrasound than that by free diffusion, indicating that ultrasound accelerated the diffusion of insulin in interstitial fluid.

### Embodiment V

Intelligent Control of Blood Glucose in Diabetic Rats with the Closed-Loop System.

The in-vivo performance of the closed-loop system was studied on STZ diabetic rats. FIG. 25(a) shows the experimental setup, including a rat with a closed-loop device on its back, a PCB and a computer. A diabetic rat was anesthetized by inhaling isoflurane, and then the closed-loop device with the microneedles was fixed on the rat′ back that was depilated.

After connecting the device to a laptop with the conducting wire, a low-glucose suspension algorithm in the software was triggered to control the closed-loop function (FIG. 23, Supporting Information). The threshold-based insulin suspension system interrupts insulin delivery when sensor glucose reaches a predefined low sensor threshold. Compared with insulin pump therapy alone, automated insulin suspension is safe and can significantly reduce the incidence and duration of severe nocturnal hypoglycemia without compromising safety, and does not increase glycated hemoglobin values.

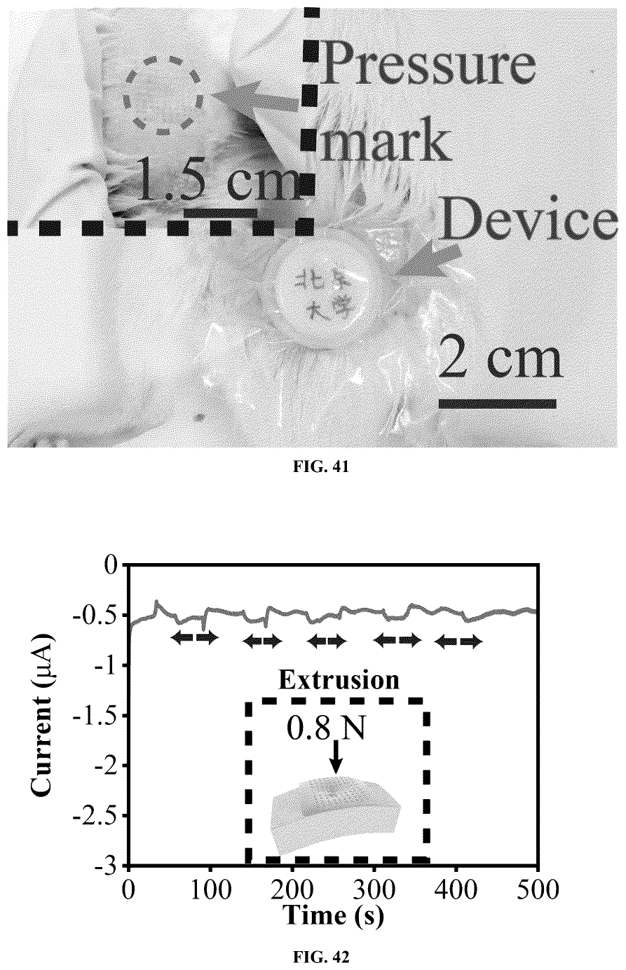

After pushing the device to the skin, the microneedles penetrated the epidermis and entered interstitial fluid in rats. The junction of dark color and light color on the skin surface was the breaking point (FIG. 40, Supporting Information). After removing the device, the penetrated pore arrays were left on the skin (FIG. 41, Supporting Information), and the microneedles did not collapse (FIG. 21, Supporting Information). The microneedle sensing device had a good electronic connection with the rat's skin (FIG. 42, Supporting Information). After applying a force of 0.8 N to the rats' skin vertically, the current changed by about 100 nA, and this was probably caused by the contact resistance change between the microneedle sensor and interstitial fluid. Such mechanical disturbance corresponded to the possible misestimation of blood glucose of 0.5 mM.

Before testing with the system, blood glucose from the rat's tail was measured by a commercial glucometer. The levels of blood glucose and the initial sensing currents were used for calibration, and a linear relationship was established. Conventional Nyquist kinetic criteria for blood glucose proved a time interval of 4-10 min as the reliable delay interval between interstitial glucose and blood glucose, but this delay would be more pronounced while blood glucose changes quickly just as the insulin injection. However, the glucose gradient would decrease as the insulin diffusion and adsorption. Thus, to further bring interstitial glucose close to blood glucose, and to avoid the noise signal caused by the injecting flow on sense, a work mode with alternating glucose sensing and insulin delivery was adopted with a cycle time of 10 min with a 1 min administration (FIG. 25(b)). For performing the insulin administration, the insulin flow rate was 120 μl/min (equivalent to an insulin content of 1.2 U/min).

After that, a predicted value of blood glucose was detected every 10 min, and a reference value of blood glucose was detected by a commercial glucometer. In the test, the injection of insulin or glucose changed the blood glucose levels of the rat dramatically. In FIG. 25(c), the sensing current exhibited the same changing trend as the blood glucose level measured by a glucometer, showing an excellent accuracy of the sensor to determine the level of blood glucose. The blood glucose level was still falling, but the current remained flat at 130 min. At 190 min, the blood glucose remained flat, but the current was still rising. The results showed that the change in current was slower than the change in glucose by approximately 10 min.

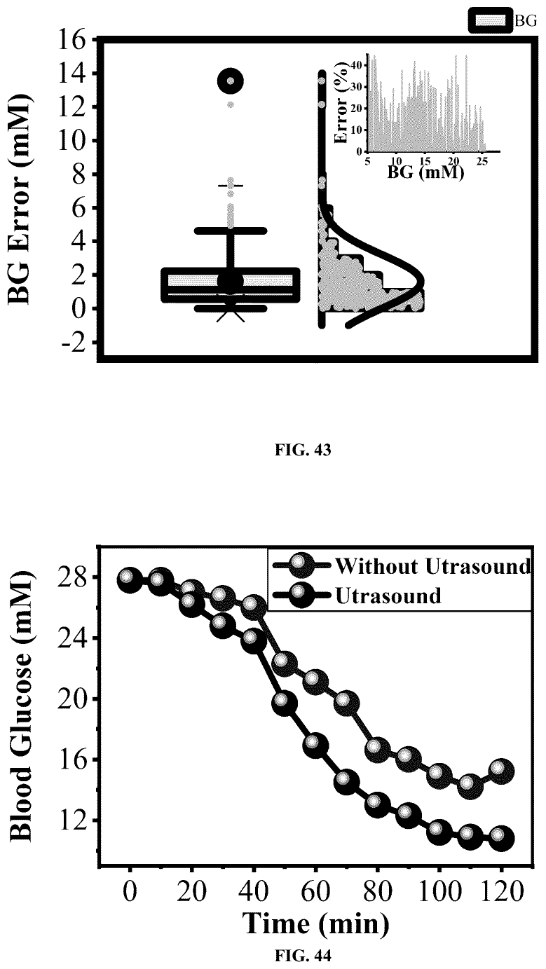

As shown in FIG. 25(d), the reference and predicted blood glucose values were analysed using the Clark error grid (CEG) that was divided into five regions (A, B, C, D and E). The device's MARD (mean absolute difference: the mean absolute relative value of the difference between the predicted and reference values divided by the reference value) was 11% for the 360 sampling sites, which demonstrated an excellent precision for the sensor. In the CEG, 100% of the measured values were located in the areas of A and B that were clinically acceptable. Among these, 86% of the measured values were in the area A, which enabled the user to make a clinical treatment plan correctly. 14% of the measured values were in the area B, which was unlikely to lead to clinically harmful treatment decisions. None of the predicted points were located in other danger zones. A statistical plot of the dispersion of glucose errors showed that a mean error was 1.4 mM, the errors for 76% of the points were less than 2 mM, and only 4% of the points were extreme outputs (FIG. 43, Supporting Information). The results indicate that the intelligent closed-loop system is accurate in predicting the blood glucose level, which is safe to use.

FIG. 25(e) shows that there was no change of blood glucose in diabetic rats with anesthesia, saline injection and microneedle insertion, indicating that other operations would not reduce blood glucose without the pump. When insulin was delivered by an ultrasonic pump, the level of the rats' blood glucose decreased gradually to 50% of the original value in two hours, which means the system could control blood glucose efficiently.

FIG. 25(f) shows the changing of blood glucose over time (BG-time curve) of the continuous subcutaneous insulin injection (CSII) with ultrasound (top), the one single injection (OSI) without ultrasound (middle) and the OSI with ultrasound (bottom). The local excessive insulin could cause a poor correlation between the levels of interstitial glucose and blood glucose. A lower predicted value was determined, since the interstitial glucose level was much less than the blood glucose level (middle figure), resulting in a wrong decision of stopping the insulin injection in hyperglycemia for treating diabetes. The acoustic cavitation, acoustic flow, and induced perforation inducted by ultrasound could accelerate the insulin diffusion to eliminate this unbalance between blood glucose and interstitial glucose. The error (orange area) between predicted and reference values were reduced when using ultrasound (top and bottom figures), compared with the free diffusion (middle figure). Also, the time spent for blood glucose to reach normoglycemia with ultrasound (bottom) was shorter than that without ultrasound (middle). For example, a 20% enhancement was achieved with ultrasound for the lowering effect of blood glucose compared that without ultrasound (FIG. 44, Supporting Information).

FIG. 25(h) shows the effect of the intelligent closed-loop system on diabetic rats on blood glucose. The system used an alternating sensing and insulin injection with a 10-min cycle time. After turning on the system, blood glucose decreased dramatically from a high level. When it reached a threshold level (<8.3 mm), the insulin delivery was stopped, and then the levels of blood glucose rebounded slightly because of the stopping of insulin. For the closed-loop device operated on a diabetic rat continuously (red line), once the blood glucose level was detected to be above the threshold value, the closed-loop device would trigger the pump to inject insulin again, and then blood glucose would decrease below the normoglycemia again. However, without the closed-loop function, after stopping insulin, blood glucose rebounded above the threshold value, owing to the possible stress response from a rapid variation of blood glucose level. Then, blood glucose still remained at dangerous hyperglycemia, as shown in the blue line of FIG. 25(h). Thus, a closed-loop system is more effective to maintain blood glucose in a safe and normal range.

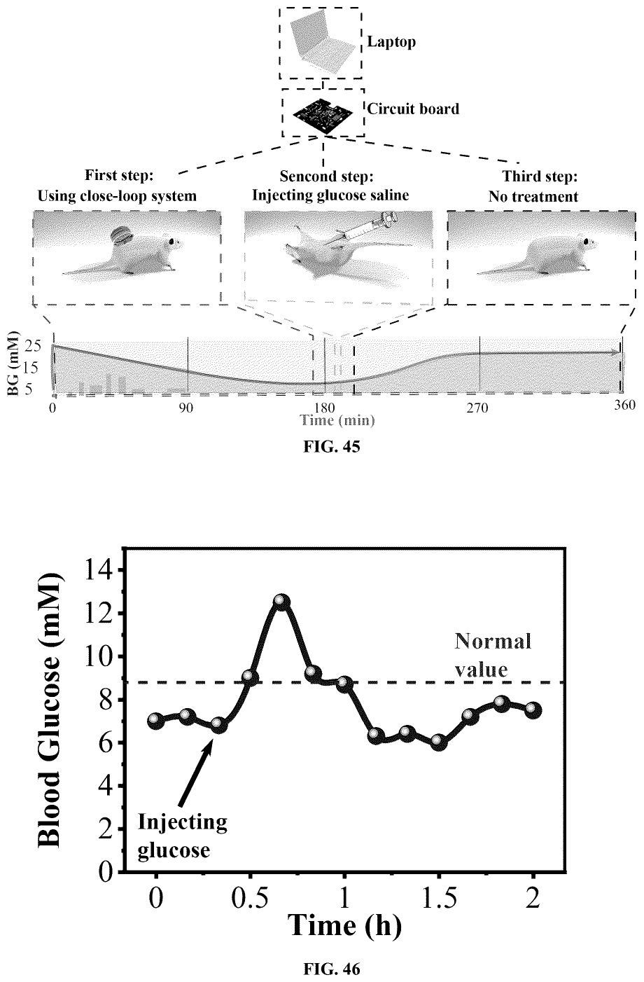

Furthermore, to evaluate the efficacy of diabetes therapies with the closed-loop system at some daily scenes such as meals, a dramatic fluctuation of blood glucose was induced by injecting glucose into diabetic rats intraperitoneally, as illustrated in FIG. 25(g). FIG. 25(i) displayed that firstly blood glucose declined to normoglycemia by the closed-loop device. Subsequently, a rat was injected intraperitoneally with a 3 ml of glucose (100 mg/ml), and then the blood glucose level increased continuously to 13-15 mM in about 60 min. In the presence of the closed-loop function by controlling blood glucose intelligently (red line), blood glucose returned to normoglycemia again within 1 hour because of the released insulin by the ultrasonic insulin pump. However, without the closed-loop function (blue line), the blood glucose level still remained at a dangerous level of 13 mM after the glucose injection. FIG. 25(i) further demonstrates that a closed-loop system worked better to control blood glucose than CSII without a closed-loop function (FIG. 45, Supporting Information) when blood glucose changed dramatically, just like with a meal. This closed-loop system is able to make the right and intelligent decisions for the daily management of diabetes.

The effect of the closed-loop system on rats with regular glucose levels was studied as well. After subcutaneous injection of glucose into normal rats (blood glucose lower than 8.3 mM), blood glucose rose rapidly within 10 min, and reached 12.5 mM after 20 min (FIG. 46, Supporting Information). Subsequently, under the regulation of the closed-loop system, the blood glucose level dropped back in time stabilized at about 7 mM, as the ultrasonic pump automatically injected insulin. These results demonstrate that the closed-loop system can effectively manage blood glucose.

The study of the rat model successfully proved the excellent ability of the ultrasonic insulin pump-enabled closed-loop patch in monitoring glucose levels and delivering insulin. As illustrated in Table 2 compared with the previously reported closed-loop system, this device has the advantages of small size, high accuracy, reusability, long management time, fast hypoglycemic rate, and a wide range of blood glucose management.

TABLE 2
Comparison of this work and other diabetes treatment systems

| | | | | | | | Device | | | | |
|---|---|---|---|---|---|---|---|---|---|---|---|
| | | Closed | | | Glucose | | of | Hypo- | Manage- | BG | |
| | | loop or | Is it | Glucose | sensing | Accuracy | delivery | glycemic | ment | range | |
| Device | Size | not | disposable | sensor | Object | (MARD) | insulin | rate | time | (mM) | Ref |
| Electro- | About | Y | Y | Film | Sweat | Correlation | Thermo- | 12 mM/ | 6 h | 7~22 | 1 |
| chemical | 100 × | | | sensor | | factor: | responsive | 4 h | | | |
| device based | 40 × 5 | | | | | 0.017 | MNS | | | | |
| on thermos- | mm | | | | | | and | | | | |
| responsive | | | | | | | heater | | | | |
| MNs | | | | | | | | | | | |
| Mesoporous | 28.5 × | Y | N | Film | ISF | 17.5 ± | Meso- | 17 mM/ | 3 h | 3~22 | 2 |
| MNs based | 42.2 × | | | sensor | extracted | 13.9% | porous | 4 h | | | |
| on | 7.7 mm | | | | out of | | MNS | | | | |
| iontophoresis | | | | | skin | | with | | | | |
| | | | | | | | ionto- | | | | |
| | | | | | | | phoresis | | | | |
| Glucose- | No | N | Y | Sensorless | No | No | Glucose- | 16 mM/ | 12 h | 6~22 | 3 |
| responsive | precise | | | | sensing | glucose | responsive | 1 h | | | |
| insulin patch | size | | | | object | sensing | insulin | | | | |
| | | | | | | | micronee | | | | |
| SiNW-FET | No | N | N | Mns | ISF | No | No | 3.8 mM/ | 1.5 h | 8~25 | 4 |
| MNs array | precise | | | array | | precise | precise | 0.5 h | | | |
| | size | | | sensor | | MARD | device | | | | |
| MNS array | Φ25 × | Y | N | Mns | ISF | 13% ± | Electro- | 12 mM/ | 5 h | 4~20 | 5 |
| with | 15 mm | | | array | | 4.11% | osmotic | 3 h | | | |
| electro- | | | | sensor | | | pump | | | | |
| osmotic pump | | | | | | | | | | | |
| Microtube | Φ20 × | Y | N | Microtube | ISF | 9.34% ± | Electro- | 14 mM/ | 4 h | 8~21 | 6 |
| with | 30 mm | | | sensor | | 6.27% | osmotic | 2.5 h | | | |
| electro- | | | | | | | pump | | | | |
| osmotic pump | | | | | | | | | | | |
| MNs array | Φ25 × | Y | N | MNs | ISF | 9.96% | Ultrasonic | 12 mM/ | 5 h | 5~23 | This |
| with | 10 mm | | | array | | | pump | 2 h | | | work |
| ultrasonic | | | | sensor | | | | | | | |
| pump | | | | | | | | | | | |

Wherein,
1 Lee, H.; Choi, T. K.; Lee, Y. B.; Cho, H. R.; Ghaffari, R.; Wang, L.; Choi, H. J.; Chung, T. D.; Lu, N. S.; Hyeon, T.; Choi, S. H.; Kim, D. H., A graphene-based electrochemical device with thermoresponsive microneedles for diabetes monitoring and therapy. Nat. Nanotechnol. 2016, 11 (6), 566-572.
2 Li, X. L.; Huang, X. S.; Mo, J. S.; Wang, H.; Huang, Q. Q.; Yang, C.; Zhang, T.; Chen, H. J.; Hang, T.; Liu, F. M.; Jiang, L. L.; Wu, Q. N.; Li, H. B.; Hu, N.; Xie, X., A Fully Integrated Closed-Loop System Based on Mesoporous Microneedles-Iontophoresis for Diabetes Treatment. Adv. Sci., 15.
3 Yu, J. C.; Wang, J. Q.; Zhang, Y. Q.; Chen, G. J.; Mao, W. W.; Ye, Y. Q.; Kahkoska, A. R.; Buse, J. B.; Langer, R.; Gu, Z., Glucose-responsive insulin patch for the regulation of blood glucose in mice and minipigs. Nat. Biomed. Eng 2020, 4 (5), 499-506.
4 Heifler, O.; Borberg, E.; Harpak, N.; Zverzhinetsky, M.; Krivitsky, V.; Gabriel, I.; Fourman, V.; Sherman, D.; Patolsky, F., Clinic-on-a-Needle Array toward Future Minimally Invasive Wearable Artificial Pancreas Applications. ACS Nano 2021, 15 (7), 12019-12033.
5 Luo, X.; Yu, Q.; Liu, Y.; Gai, W.; Ye, L.; Yang, L.; Cui, Y., Closed-Loop Diabetes Minipatch Based on a Biosensor and an Electroosmotic Pump on Hollow Biodegradable Microneedles. ACS Sens. 2022, 7 (5), 1347-1360.
6 Liu, Y.; Yu, Q.; Luo, X.; Ye, L.; Yang, L.; Cui, Y., A Microtube-Based Wearable Closed-Loop Minisystem for Diabetes Management. Research 2022, 2022.

In the description of the present disclosure, it needs to be illustrated that the indicative direction or position relations of the terms such as “center”, “top”, “bottom”, “left”, “right”, “vertical”, “horizontal”, “inside” and “outside” are direction or position relations illustrated based on the attached figures, just for facilitating the description of the present disclosure and simplifying the description, but not for indicating or hinting that the indicated device or element must be in a specific direction and is constructed and operated in the specific direction, the terms cannot be understood as the restriction of the present disclosure. Moreover, the terms such as “first” and “second” are just used for distinguishing the description, but cannot be understood to indicate or hint relative importance.

Specific examples are used for illustration of the principles and implementation methods of the present disclosure. The description of the above-mentioned embodiments is used to help illustrate the method and its core principles of the present disclosure. In addition, those skilled in the art can make various modifications in terms of specific embodiments and scope of application in accordance with the teachings of the present disclosure. In summary, the contents of this specification should not be understood as the limitation of the present disclosure.

What is claimed is:

1. An insulin injection pump, comprising an upper housing and a lower housing, wherein a film is arranged between the upper housing and the low housing, an insulin storage chamber is formed between the film and the upper housing, several conical holes are formed in the film, large-diameter ends of the conical holes are adjacent to the insulin storage chamber, a piezoelectric ring is arranged on a side of the film facing away from the insulin storage chamber, edges of two opposite sides of the film and the piezoelectric ring are connected with an external alternating current power supply through wires, respectively, and a liquid outlet is formed in the lower housing.

2. The insulin injection pump according to claim 1, wherein the film is made of hard film materials or flexible film materials.

3. The insulin injection pump according to claim 1, wherein the piezoelectric ring is made of piezoelectric crystals, piezoelectric ceramics or piezoelectric polymers.

4. The insulin injection pump according to claim 1, wherein insulin, with a concentration of 1-500 U/ml, is stored in the insulin storage chamber.

5. The insulin injection pump according to claim 1, wherein sealing rings are arranged between the film and the upper housing and between the piezoelectric ring and the lower housing; and a liquid inlet is formed in the upper housing, and a rubber plug is arranged at the liquid inlet.

6. A manufacturing method of an insulin injection pump, comprising the following steps:
S1, preparing a film: taking a thin sheet with a thickness of 1-5 mm, and forming conical holes by laser etching or ion selective etching therein;
S2, preparing a piezoelectric ring: selecting piezoelectric materials, and performing sputtering deposition of a layer of piezoelectric materials through mask holes at an outer edge of the film to form a ring which is integrated with the film and has a same outer diameter as an outer diameter of the film;
S3, preparing an upper housing: integrally molding an upper housing through 3D (three-dimensional) printing or injection molding;
S4, preparing a lower housing: integrally molding a lower housing through 3D printing or injection molding; and
S5, assembling: sequentially placing a sealing ring, the film, the piezoelectric ring and a sealing ring in the upper housing to seal and connect the lower housing with the upper housing.

7. A closed-loop system, comprising a sensor, a control unit and the insulin injection pump according to claim 1, wherein the sensor comprises electrodes, and the sensor is arranged at a position of a liquid outlet of the insulin injection pump; an input end of the control unit is connected with an output end of the sensor, and an output end of the control unit is connected with an input end of the insulin injection pump; and the control unit controls a start and stop of the insulin injection pump after receiving an electrical signal from the sensor.

8. The closed-loop system according to claim 7, wherein the sensor is a hose sensor, the hose sensor comprises a tubular base, and the electrodes are circumferentially distributed on an outer wall of the base; a cross section of the tubular base is circular or polygonal, and a length of the tubular base is 1 mm to 15 mm.

9. The closed-loop system according to claim 7, wherein the sensor is a microneedle biosensor, the microneedle biosensor comprises a substrate, a microneedle array is integrally formed on the substrate, the microneedle array comprises several hollow microneedles, and the substrate is covered with the electrodes; and each of the several hollow microneedles is conical or pyramid-shaped, and a large-diameter end of each of the several hollow microneedles is connected with the substrate.

10. The closed-loop system according to claim 7, wherein the control unit comprises a signal acquisition module, a control module and an execution module, the signal acquisition module is configured for receiving an electrical signal from the sensor and converting the signal; the control module analyzes the electrical signal collected by the signal acquisition module and issues instructions to the execution module; and the execution module controls the start and stop of the insulin injection pump according to the instructions sent by the control module.
